(19) United States
(12) Patent Application Publication
(10) Pub. No.: US 2021/0333265 A1
(43) Pub. Date: Oct. 28, 2021

(54) MINI-CANCERS UTILIZATION FOR PERSONALIZED CANCER DRUG REGIMENS

(71) Applicant: THE JOHNS HOPKINS UNIVERSITY, Baltimore, MD (US)

(72) Inventors: Florin Selaru, Catonsville, MD (US); Ling Li, Baltimore, MD (US)

(21) Appl. No.: 16/479,652

(22) PCT Filed: Feb. 1, 2018
(86) PCT No.: PCT/US2018/016356

§ 371 Date: Jul. 22, 2019

Related U.S. Application Data
(60) Provisional application No. 62/454,133, filed on Feb. 3, 2017.
(60) Provisional application No. 62/468,975, filed on Mar. 9, 2017.

(51) Int. Cl. G01N 33/50; G01N 33/574

(52) U.S. Cl. CPC G01N 33/5067; G01N 33/57438; G01N 33/5091; G01N 33/5011

(57) ABSTRACT

The present invention includes methods for identifying chemotherapy treatments specifically suited for each cancer patient using mini-cancers to develop personalized cancer drug regimes.

### REFERENCE TO RELATED APPLICATIONS

This application claims the benefit of U.S. Provisional Patent applications 62/454,133, filed Feb. 3, 2017, and 62/468,975, filed Mar. 9, 2017, which are hereby incorporated by reference for all purposes as if fully set forth herein.

### BACKGROUND OF THE INVENTION

Liver cancer has a global incidence of approximately 850,000 cases and represents the second leading cause of cancer-related mortality. Hepatocellular carcinoma (HCC) accounts for 80-85% of all cases of primary liver cancers. Cholangiocarcinoma (CCA) is a primary liver cancer with a median survival after diagnosis of less than 12 months. Gastric cancer is the third leading cause of cancer death in the world. The paucity of Food and Drug Administration (FDA) approved drugs for these cancers contributes at least in part to this dismal survival. It is possible that alternative drugs that are FDA approved for a different indication are superior to the current standard of care in these cancers. There are, however, logistical limitations to performing standard clinical trials to expand the indication of a currently FDA approved cancer drug (accruing sufficient numbers of patients, associated cost, as well as identification of a payer for such clinical trials). In addition, it is plausible that a cancer patient may uniquely respond to a chemotherapy agent that would not be effective for other cancer patients. However, a standard, reproducible and translatable pipeline to rapidly test FDA approved drugs for a specific patient does not exist and is currently needed.

### SUMMARY OF THE INVENTION

One embodiment of the present invention is a method of identifying a chemotherapy treatment for a subject having cancer comprising: obtaining a biopsy from a subject having cancer; establishing a cancer culture from the biopsy; mixing the cancer culture with a matrix forming mini cancers; growing the mini cancers; dividing the mini cancers into one or more samples; adding one or more agents separately or combined to the one or more samples; analyzing the phenotypes of the mini cancers wherein the phenotypes are selected from the group of cell growth, invasion, or a combination thereof; and identifying a chemotherapy treatment for the subject by choosing the one or more agents determined to stop growth of the mini cancers, inhibit growth of the mini cancers, and/or inhibit invasion of the mini cancers. Potentially all cancers maybe used in the present invention including liver, gastric (stomach) and pancreatic cancers. For example, if a subject has liver cancer, then a cancer culture maybe created with the biopsy of a cancerous liver and mixing it with an establishing growth media comprising: HGF, FSK, FSK-10, Gastrin 1, NAC B27, N2, Nicotinamide, Wnt3A, R-spondin1, EGF, Noggin, A83-01, FGF2, PGE2, and Y27632. Then a cancer culture of the present invention maybe grown with a second growth media comprising: HGF, FSK, FGF10, Gastrin I, NAC, B27 supplement, N2, Nicotinamide, Wnt3A-conditioned medium, R-spondin1, EGF, Noffin conditioned medium, A83-01, FGF2, and PGE2. For gastric (stomach) cancer, it is necessary to remove HGF, FGF2, FSK and FGF10 and add PGE2 to the standard media above. With some cancers, such as a desmoplastic cancer, for example, cells selected from the group comprising fibroblasts, endothelial cells, immune cells, other tumor-specific cells, or combinations thereof, are added to the cancer culture. Potentially all matrigel are suitable for the present invention including those that have a polymer such ascollagen, gelatin, chitosan, heparin, fibrinogen, hyaluronic acid, chondroitin sulfate, pullulan, xylan, dextran, polyethylene glycol, derivatives thereof, or a combination thereof. The step of identifying a chemotherapy treatment of the present invention maybe based on a percentage of mini cancers killed by the agent when compared to a reference culture comprising mini cancers substantially free of the one or more agents; a percentage of mini cancers having inhibited growth when compared to a reference culture comprising mini cancers substantially free of the one or more agents; a percentage of mini cancers that are inhibited from invasion when compared to a reference culture comprising mini cancers substantially free of the one or more agents; or a combination thereof. The methods of the present invention may include many samples, for example, in the range of 10 to 4,000 samples.

Another embodiment of the present invention is a cell culture comprising HGF, FSK, FSK-10, Gastrin 1, NAC B27, N2, Nicotinamide, Wnt3A, R-spondin1, EGF, Noggin, A83-01, FGF2, PGE2, and Y27632.

Another embodiment of the present invention is a cell culture comprising: HGF, FSK, FGF10, Gastrin I, NAC, B27 supplement, N2, Nicotinamide, Wnt3A-conditioned medium, R-spondin1, EGF, Noffin conditioned medium, A83-01, FGF2, and PGE2.

Another embodiment of the present invention is a method of treating a patient having cancer comprising: obtaining a cancer fragment/biopsy from a subject; establishing a cancer culture from the biopsy; mixing the cell culture with a matrix forming mini cancers; growing the mini cancers; dividing the mini cancers into one or more samples; adding one or more agents separately, or combined, to the one or more samples; analyzing the phenotypes of the mini cancers wherein the phenotypes are selected from the group of cell growth, invasion, or a combination thereof; identifying a chemotherapy agent for the subject by choosing the one or more agents determined to stop growth of the mini cancers, inhibit growth of the mini cancers, or inhibit invasion of the mini cancers; and administering the one or more agents to the subject to treat or prevent the cancer. The methods of the present invention may be repeated should the subject have a cancer recurrence after treatment and the subject maybe treated with a second one or more agent identified during the repeated process. Potentially any cancer may be used in the present invention. For example, if the cancer is liver cancer, then a cancer culture is created with the biopsy of a liver, and a cancer culture of the present invention may be mixed with an establishing growth media comprising: HGF, FSK, FSK-10, Gastrin 1, NAC B27, N2, Nicotinamide, Wnt3A, R-spondin1, EGF, Noggin, A83-01, FGF2, PGE2, and Y27632. Then a cancer culture of the present invention is grown on a second growth media comprising: HGF, FSK, FGF10, Gastrin I, NAC, B27 supplement, N2, Nicotinamide, Wnt3A-conditioned medium, R-spondin1, EGF, Noffin conditioned medium, A83-01, FGF2, and PGE2. When certain cancers are used in the present invention, such as to desmoplastic cancer, for example, cells selected from the group comprising fibroblasts, endothelial cells, immune cells, other tumor-specific cells, or combinations thereof are added to a cancer culture. As mentioned above, matrigels used in the present invention may comprises a polymer selected from the group consisting of collagen, gelatin, chitosan, heparin, fibrinogen, hyaluronic acid, chondroitin sulfate, pullulan, xylan, dextran, and polyethylene glycol as well as their derivatives or a combination thereof.

Another embodiment is the identification of 9 pan-effective agents for liver cancers. These agents/drugs were identified by applying the processes described here on a cohort of primary liver mini-cancers. These agents are: HDAC inhibitors (romidepsin, panobinostat); proteasome inhibitors (ixazomib, bortezomib and carfilzomib), DNA topoisomerase II inhibitors (idarubicin, daunorubicin and topotecan), RNA synthesis inhibitor (Plicamycin) or combinations thereof.

Another embodiment is the creation of complex mini-cancers. These complex mini cancers contain epithealial cancer cells, as well as other cell types found in human tumors in vivo (such as fibroblasts and/or endothelial cells, as examples). The inventors have shown that primary human mini-cancers (that would be sensitive to drugs when cultured on their own) become resistant. These findings mirror the experience with Phase III clinical trials, whereby compounds found to work in preclinical models fail in human Phase III trials. The constructs that were built help explain at least in part why this happens, can help prevent unnecessary Phase III trials as well as allow identification of compounds to inhibit stroma.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “activity” refers to the ability of a gene to perform its function such as Indoleamine 2,3-dioxygenase (an oxidoreductase) catalyzing the degradation of the essential amino acid tryptophan (trp) to N-formyl-kynurenine.

The term “agent” refers to any small molecule chemical compound, antibody, nucleic acid molecule, or polypeptide, or fragments thereof.

The term “ameliorate” refers to decrease, suppress, attenuate, diminish, arrest, or stabilize the development or progression of a disease.

The term “alteration” refers to a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

The term “analog” refers to a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

The term “CCA” refers to Cholangiocarcinoma.

The term “diagnostic” refers to identifying the presence or nature of a pathologic condition, i.e., pancreatic cancer. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The term “disease” refers to any condition or disorder that damages or interferes with the normal function of a cell, tissue, or organ. Examples of diseases include cancer.

The term “effective amount” refers to the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “express” refers to the ability of a gene to express the gene product including for example its corresponding mRNA or protein sequence(s).

The term “HCC” refers to Hepatocellular carcinoma.

The term “hybridization” refers to hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The term “immunoassay” refers to an assay that uses an antibody to specifically bind an antigen (e.g., a marker). The immunoassay is characterized by the use of specific binding properties of a particular antibody to isolate, target, and/or quantify the antigen.

The term “marker” refers to any protein or polynucleotide having an alteration in expression level or activity that is associated with a disease or disorder. The term “biomarker” is used interchangeably with the term “marker.”

The term “measuring” refers to methods which include detecting the presence or absence of marker(s) in the sample, quantifying the amount of marker(s) in the sample, and/or qualifying the type of biomarker. Measuring can be accomplished by methods known in the art and those further described herein, including but not limited to immunoassay. Any suitable methods can be used to detect and measure one or more of the markers described herein. These methods include, without limitation, ELISA and bead-based immunoassays (e.g., monoplexed or multiplexed bead-based immunoassays, magnetic bead-based immunoassays).

The term, “obtaining” as in “obtaining an agent” refers to synthesizing, purchasing, or otherwise acquiring the agent.

The term “PDO” refers to a patient derived organoid.

The terms “polypeptide,” “peptide” and “protein” are used interchangeably herein and refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

The term “reduces” refers to a negative alteration of at least 10%, 25%, 50%, 75%, or 100%.

The term “reference” refers to a standard or control conditions such as a sample (such as minicancers) or a subject that is a free, or substantially free, of an agent such as one or more chemotherapy agents, as an example.

The term “reference sequence” refers to a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

The term “sensitivity” refers to the percentage of subjects with a particular disease.

The term “specificity” refers to the percentage of subjects correctly identified as having a particular disease i.e., normal or healthy subjects. For example, the specificity is calculated as the number of subjects with a particular disease as compared to non-cancer subjects (e.g., normal healthy subjects).

The term “specifically binds” refers to a compound or antibody that recognizes and binds a polypeptide of the invention, but which does not substantially recognize and bind other molecules in a sample, for example, a biological sample, which naturally includes a polypeptide of the invention.

The term “subject” refers to any individual or patient to which the method described herein is performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

The terms “prevent,” “preventing,” “prevention,” “prophylactic treatment” and the like refer to reducing the probability of developing a disorder or condition in a subject, who does not have, but is at risk of or susceptible to developing a disorder or condition.

Such treatment (surgery and/or chemotherapy) will be suitably administered to subjects, particularly humans, suffering from, having, susceptible to, or at risk for pancreatic cancer or disease, disorder, or symptom thereof. Determination of those subjects “at risk” can be made by any objective or subjective determination by a diagnostic test or opinion of a subject or health care provider (e.g., genetic test, enzyme or protein marker, a marker (as defined herein), family history, and the like).

### BRIEF DESCRIPTION OF THE DRAWINGS

FIG. 1 illustrates a road map for precision drug selection.

FIG. 2 illustrates an establishment of Patient Derived organoid (PDO) from CCA.

FIG. 3 illustrates an epithelial, stem, and biliary markers.

FIG. 4 illustrates that cisplatin has no effect on the round shape of PDO. Vinorelbine is effective.

FIG. 5 illustrates that Cisplatin is ineffective, Gemicitabine partially effective and Bortezomib is effective.

FIG. 6 illustrates red highlights effective and blue ineffective drugs.

FIG. 7 illustrates IC50 curves for 6 drugs on 5 CCA PDO specimens. The killing effect was read with ATP-ase CellTiter-Glo (Promega).

FIG. 8 illustrates Ponatinib inhibits FGFR1 mutated PDOs only at high concentrations.

FIG. 9 illustrates vital stains-DRAQ5 and YOYO-1.

FIG. 10 illustrates H & E Stain of CCA PDX (100X0.

FIG. 11 illustrates complex PDO:CCA Organoids and myofibroblasts.

FIG. 12 illustrates complex PDO: Red arrows indicate expanding CCA PDO growing in a matrix containing fibroblasts and endothelia cells.

FIG. 13 illustrates an embodiment of a drug testing schematic.

FIG. 14 illustrates a study of the effects of passaging CCA PDO on drug response profiles.

FIG. 15 illustrates four types of complex PDO constructs.

FIG. 16 illustrates a chart comparing cancer models.

FIG. 17 illustrates the identification of 9 pan-effective drugs based on screening a large number of drugs on a large cohort of primary liver mini-cancers

FIG. 18 illustrates a patient derived xenograft (PDX) established from a primary liver mini-cancer. This model can be used to verify predictions in mini-cancers, since PDX models mirror human response to drugs.

FIG. 19 illustrates the effect of Panobinostat on simple and complex minicancers. The figure shows stromal cells (lower panels, in red) keep cancer cells alive (round, green mini-cancers). The figure also shows how the IC50 curve shifts, demonstrating the powerful drug-resistance effects of stromal cells and quantifying this effect

FIG. 20 illustrates how stromal cells impact the sensitivity of mini-cancers to carfilzomib.

FIG. 21 illustrates how stromal cells impact the sensitivity of mini-cancers to bortezomib.

### DETAILED DESCRIPTION OF THE INVENTION

Liver cancer has a global incidence of approximately 850,000 cases and represents the second leading cause of cancer-related mortality. Hepatocellular carcinoma (HCC) accounts for 80-85% of all cases of primary liver cancers. Cholangiocarcinoma (CCA), the second most prevalent primary liver cancer, has a median survival after diagnosis of less than 12 months. Next generation sequencing (NGS) approaches identified a significant number of somatic mutations in liver cancers. These strategies led to the postulate that the identification of actionable mutations will guide the selection of targeted drugs aimed at those specific mutations which in turn will result in patient- and cancer-specific therapeutics and better clinical outcomes. By inference, if each cancer had actionable mutations and if a targeted therapy existed for each, then cancer genomics could inform drug selection in all cases. However, only 3-6% of cancer patients who have their cancer exome sequenced are paired with a drug (http://ecog-acrin.org/nci-match-eay131/interim-analysis). Specific to liver cancers, multiple somatic alterations were identified in HCC, but the vast majority of molecular/targeted agents tested to date failed. Sorafenib, a multi-kinase inhibitor approved for HCC improves survival by a mere 3 months at the cost of severe side effects. Similar to clinical outcomes in HCC, in spite of NGS efforts to elucidate molecular pathways in CCA, the first line and mainstay of CCA chemotherapy continues to be Gemcitabine and Cisplatin (with a poor response rate).

The lack of effective therapeutics in liver cancers and the high failure rate of the vast majority of drugs to date led some to advance that HCC is not druggable. These efforts to identify actionable mutations or druggable pathways in liver cancers appeared to suffer an additional setback once intratumor heterogeneity was proven. First recognized in primary renal carcinomas, the phenomenon was later recognized in most solid tumors, including HCC and CCA. The implication of these studies is that intratumor genetic heterogeneity leads to a drug response/functional, intratumor heterogeneity that is clinically relevant. The importance of demonstrating this tenet cannot be overemphasized, since it may offer an explanation for at least some of the numerous therapeutic failures to date. In addition, if genetic heterogeneity is translated into drug response heterogeneity in liver cancers, then logical approaches can be devised to account for this phenomenon, perhaps through designing combinations of drugs that are effective on each clinically significant intra-tumor cancer cell population. To our knowledge, however, there is no data in liver cancers to demonstrate that genetically defined intra-tumor phylogenetic groups translate into identification of discreet drugs that are effective on each group.

Functional diagnostics describes a category of techniques to enable the experimental testing of a drug or set of drugs on live cancer cells obtained from a patient, with the goal of informing drug choice. While precision oncology is oftentimes equated to NGS of cancer exomes, the incomplete understanding of the genotype to phenotype relationship poses significant clinical translatability challenges. Carefully characterized functional diagnostics approaches bring the promise of addressing this limitation. The American Society of Clinical Oncology (ASCO), however, found that first generation functional diagnostics—Chemotherapy Sensitivity and Resistance Assays (CSRA)—have not been successful in surpassing published reports of clinical trials. These efforts, however, were restricted by a number of limitations, including lack of standardization in regards to the study design, type of assay, reproducibility and accuracy of such assays, number of drugs used (typically low number of drugs), or type of drugs used (typically standard chemotherapy)¹³. Issues such as assay validation, reproducibility and more importantly clinical translatability were incompletely and heterogeneously described. However, next generation functional diagnostics—including patient derived organoids (PDO) could address these concerns.

Functional diagnostics describes a category of techniques to enable the experimental testing of a drug or set of drugs on liver cancer cells obtained from a patient, with the goal of informing the drug choice. While precision oncology is oftentimes equated to genomics, the incomplete understanding of the genotype to phenotype relationship poses significant clinical translatability challenges. Carefully characterized functional diagnostics approaches bring the promise of addressing this limitation. Of note, the idea of matching drugs to patients has been validated in other clinical fields, such as in infectious diseases where choice of best antibiotic is guided by functional testing and not by site of infection. In oncology, The American Society of Clinical Oncology (ASCO) found that first generation functional diagnostics—Chemotherapy Sensitivity and Resistance Assays—have not been successful in surpassing published reports of clinical trials. These efforts, however, were not standardized in regards to the study design, type of assay, reproducibility and accuracy of such assays, number of drugs used (typically low number of drugs), or type of drugs used (typically standard chemotherapy). Issues such as assay validation, reproducibility and more importantly clinical translatability were incompletely and heterogeneously described. However, next generation functional diagnostics—including patient derived organoids (PDO), have been recently developed and they could address these concerns. The inventors propose to implement state-of-the-art patient-derived cancer models with the purpose of validating our existing high throughput (HT) functional diagnostics pipeline in CCA. The deliverable is a comprehensively vetted algorithm for precision functional diagnostics that is accurate, reproducible, relatively inexpensive and clinically useful.

Cancer genomics postulates that the identification of actionable mutations will guide the selection of targeted drugs aimed at those specific mutations. By extension, this method is correlative, not based on direct, experimental verification of drug effects. By inferential reasoning, if each cancer had actionable mutations and if a targeted therapy existed for each, then cancer genomics could inform drug selection in all cases. However, only 3-6% of cancer patients who have their cancer exome sequenced are paired with a drug (http://ecog-acrin.org/nci-match-eay131/interim-analysis). The chief reasons are our incomplete knowledge of these “actionable mutations” as well as the paucity of targeted drugs for such mutations. Another concern is that outcomes for the few patients who are paired to a targeted drug are not dramatically better. Drug choice based on biomarkers improved progression-free survival by only 5.7 months. In conclusion, the low numbers of patients for whom a targeted drug is identified coupled with the relatively low response rate creates an imperative for direct, experimentally verifiable precision-oncology approaches. The relevant clinical question is how to utilize the information from genome-based diagnostics as a part of a larger approach, not as the sole guide. Experiments the inventors propose will correlate and integrate drug functional testing with genomic profiling, therefore elevating the significance of our application through its clinical impact.

Patient Derived Xenografts (PDX) represent the gold standard for recapitulating the biology of a patient's cancer, including stable genomic, transcriptomic and proteomic profiles across many passages. Of clinical interest, PDX also predict drug responses in patients from whom they were derived. According to some PDX experts, this model provide in excess of 80% positive predictive value for drug activity in patients. If there were no barriers to use, PDX would be the ideal assays to inform cancer drug selection. Unfortunately, PDX display a number of limitations to their widespread, systematic use in clinical oncology. For example, PDX are labor-intensive, expensive and slow to develop (up to 10 months to establish the first engraftment), with low and/or variable take rate. High costs limit the number of drugs that can realistically be tested. PDX also induce loss of clonal heterogenetity due to mouse selection pressure, replacement of the human stromal component with mouse cells, and the lack of an immune system. Given the high accuracy of PDX, the relevant question is how to develop surrogates to parallel PDX in an accurate, reproducible, rapid and cost conscious fashion. The experiments the inventors propose will benchmark patient-derived organoids (PDO) against PDX and will clarify precisely when and how to use PDO and at which step to rely on PDX.

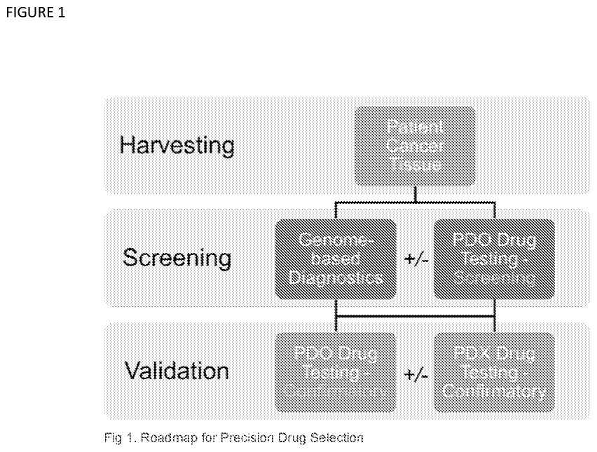

First, the inventor's findings bring them closer to a functional drug testing trial in CCA patients, who desperately need effective therapies. Second, the inventors are develop principles widely applicable in designing similar trials in other cancers. Third, the inventor's are modeling the stromal contribution to drug responses and determining the optimal assay complexity, which is of high interest to other desmoplastic cancers, such as pancreatic, prostate and subsets of breast cancer. Fourth, the inventor's are delineating the merits of current precision modalities—genomic-based diagnostics and PDX. Finally, the inventor's are determining a clinically feasible strategy for initial drug screening (genome based diagnostics vs. PDO functional diagnostics) and validation (PDO models to include fibroblasts and endothelial cells vs. PDX, see FIG. 1.

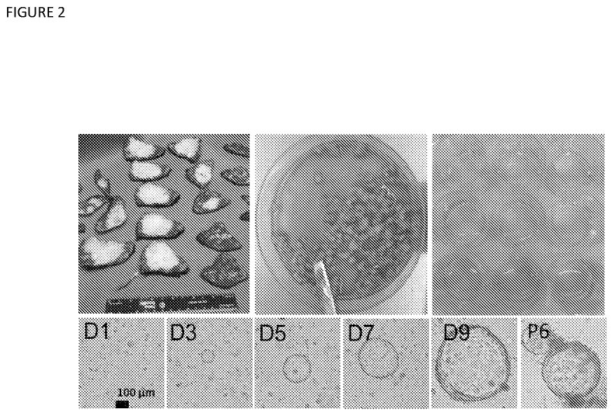

The inventors have established PDO cultures from 10/10 CCA patients seen at the Johns Hopkins Hospital (FIG. 2); Clarified conditions to successfully maintain CCA PDO in culture by adapting published normal liver protocols; Adapted experimental conditions for HT drug screening; Validated PDO assays reflective of drug efficacy (bright field, vital stains and ATP based); Successfully screened the NCI Approved Oncology Drugs Set VII (129 FDA approved cancer drugs); Successfully screened the Johns Hopkins Clinical Compound Library (JHCCL—1,600 drugs—see letters of collaboration from Dr. Slusher and Dr. Liu); Performed DNA whole exome sequencing on a subset of PDO cultures; Correlated presence of actionable mutations to experimentally verified response to targeted therapies; Performed RNA sequencing on same subset of PDO cultures; Identified a transcriptomics signature predictive of CCA response to classic (non-targeted) drugs; Performed half maximal inhibitory concentration (IC50) drug testing on lead drugs for each PDO; Created 2 distinct models of Complex PDO to include fibroblasts and endothelial cells; Completed the whole pipeline in a clinically relevant and applicable period of 6 weeks for each CCA.

The inventors are exploring three critically important features: (1) The ability of CCA PDO to maintain characteristics of original tumor. There is a pressing need to authenticate cancer PDO as key resources to be used in scientific and clinical applications; (2) The ability to assess drug efficacy through accurate, direct, assays as well as molecular profiling. The inventors will contrast several imaging modes to quantify cancer phenotypes (growth; invasion) at high accuracy and acceptable cost. In addition, the inventors will investigate the possibility that responses to specific drugs (such as mutation targeted therapies) can be predicted by genomic-based diagnostics without recourse to functional diagnostics; (3) The ability of various PDO constructs (simple and/or complex) to inform drug selection. The inventors are assessing if they can use simple PDO for initial drug screen, or do they have to use more resource-intensive cPDO (that include fibroblasts and endothelial cells. Furthermore, the inventors plan to clarify the best cPDO to parallel PDX in terms of drug selection positive predictive value.

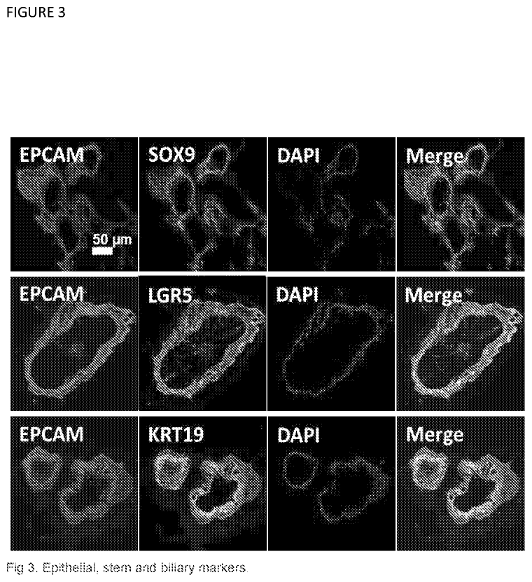

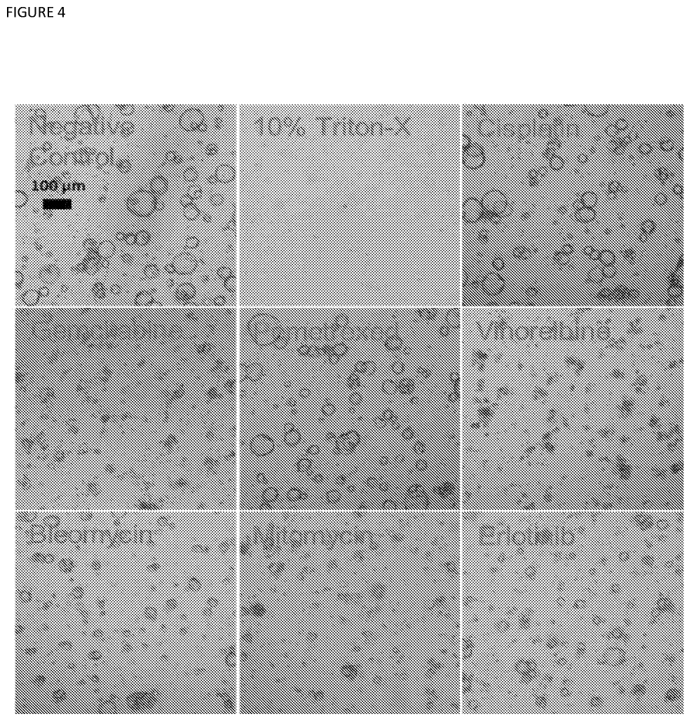

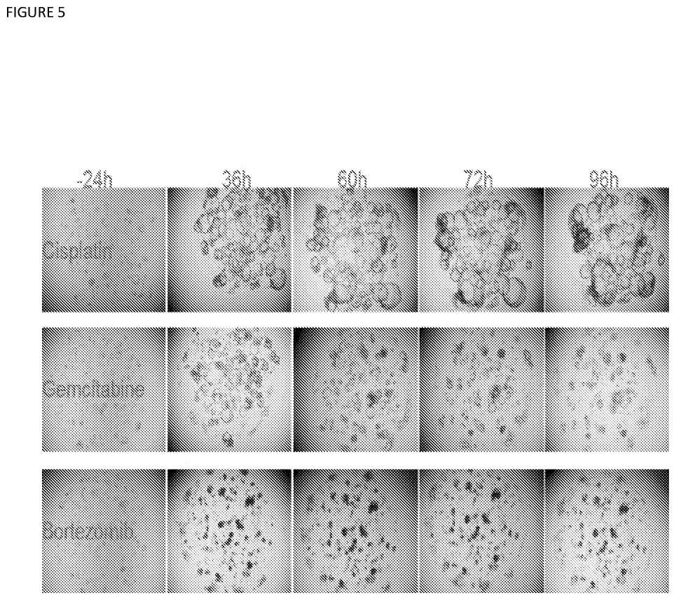

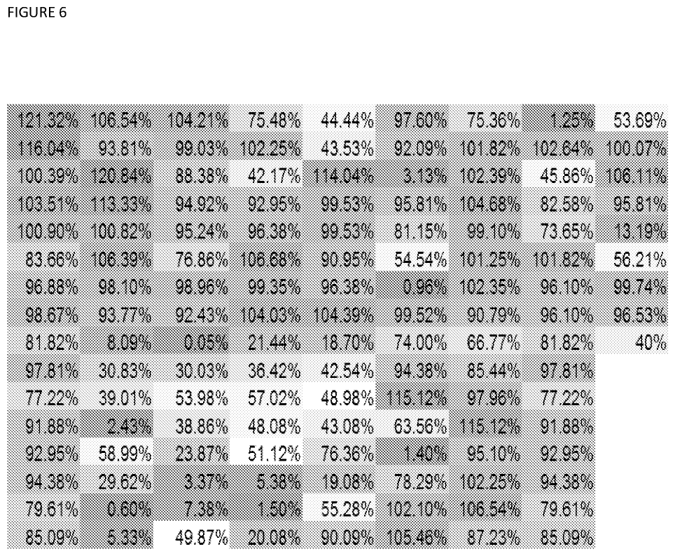

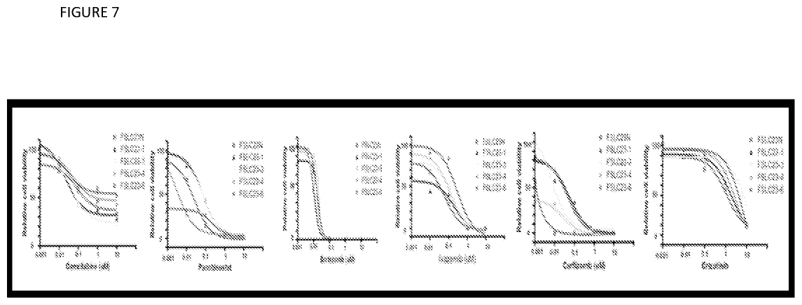

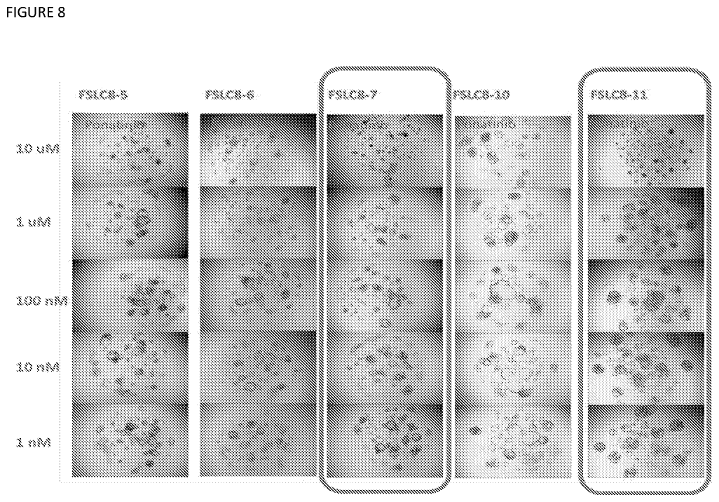

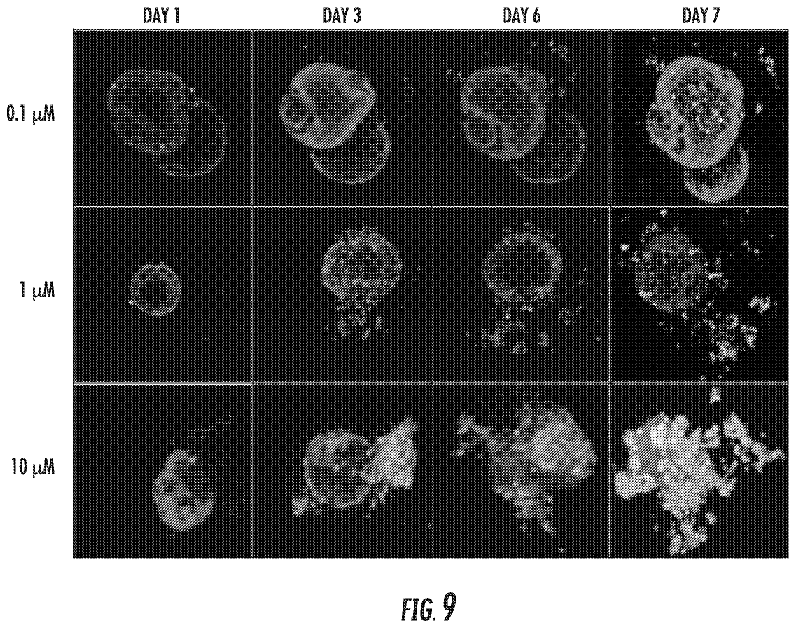

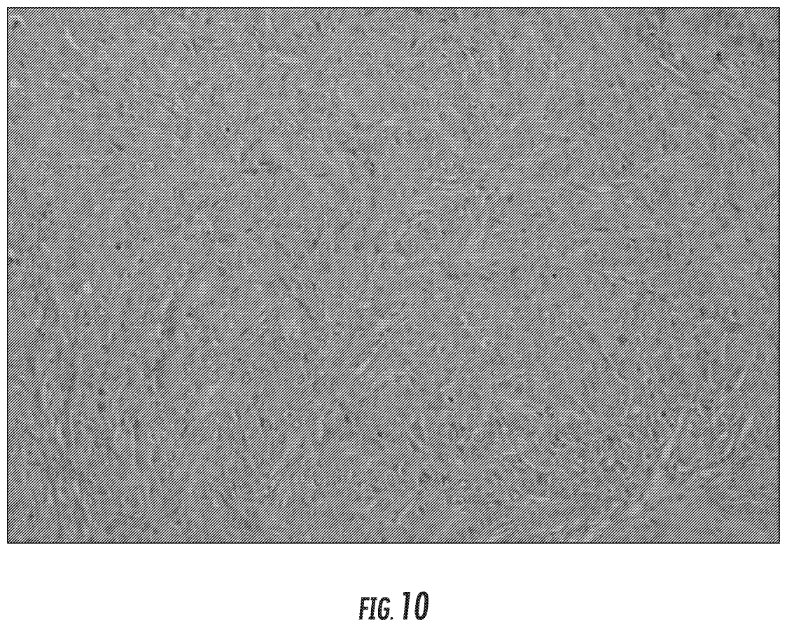

In preliminary studies, the inventors have obtained resection cancer tissue from 10 CCA patients. The inventors successfully established all 10 in patient-derived organoids (PDO) by adapting culture conditions reported for normal liver (19). As shown in FIG. 2, human CCA tumors are cut into pieces, dissociated with collagenase, and placed in Matrigel or hydrogel covered by growth media. Representative cancer PDO are shown at days 1, 3, 5, 7 and 9 as well as at a later passage. As shown in FIG. 3, PDO cultures appropriately expressed epithelial (EPCAM), stem cell (LGR5 and SOX9) and biliary epithelium markers (KRT19). Pilots experiments were performed with a 9-drug library, at a standard HT screening drug concentration of 10 uM. For positive control, the inventors used 10% Triton X, while for negative control the inventors used DMSO at same concentration used to dissolve drugs. As shown in FIG. 4, the inventors found that Cisplatin (a first line CCA drug) has no effect on this CCA. Gemcitabine (the other commonly used CCA drug) appeared to have a moderate cytostatic but not a cytostatic effect. Of note, this patient was treated with Gemcitabine and Cisplatin with a modest response. Interestingly, Vinorelbine (a drug approved for lung cancer) appeared to be effective (FIG. 4). The NCI Approved Oncology Drugs Set VII (129 FDA approved cancer drugs) were then tested on CCA PDO. Bortezomib (a first-generation proteasome inhibitor) was highly active for one CCA (FIG. 5), with the early cytocidal effect sustained over 96 hours. In contrast, Cisplatin allowed unaffected PDO growth. Gemcitabine displayed a moderate cytostatic, but not cytocidal effect, supporting the inventors' global hypothesis that FDA approved drugs already exist that would be more effective than standard of care for CCA patients. Moreover, Cisplatin was found ineffective in all 6 CCAs tested. As proof of concept, the inventors then expanded to the 1,600 drug JHCCL Hopkins library (data not shown). The inventors were able to screen the large library in a reproducible fashion on a subset of CCA PDOs. This scale-up required a reproducible and accurate read out. We chose CellTiter-Glo (Promega), an ATP-based cytotoxicity assay that quantifies metabolically active cells. The inventors have determined the Z-factor to be 0.84 which is excellent for HT drug screening. The relative cell viability for 137 drugs at 10 uM (composed of the NCI-129 drug library to which we added other 8 drugs) is shown in FIG. 6. Note that the vast majority of drugs have little to no impact on viability (blue colored boxes) while 9 drugs (6.5%) demonstrated excellent killing activity (less than 5% metabolically active cells). Next, the inventors sought to determine IC50s. For the top active drugs, the inventors performed drug testing at 10 uM, 1 uM, 100 nM, 10 nM and 1 nM. FIG. 7 demonstrates representative IC50 curves for 6 drugs tested on 5 CCA PDOs. Next, the inventors performed whole exome sequencing on a subset of these CCA PDOs. The inventors found that 2 CCA PDOs (FSLC8-7 and FSLC8-11 in the FIG. 8) displayed FGFR1 mutations while 3 others did not. This genetic information enabled the inventors to predict response to FGFR1 targeted therapy. To experimentally validate these predictions, the inventors utilized Ponatinib (tyrosine kinase inhibitor active on FGFR1 mutations, approved for some forms of leukemias). As shown in FIG. 8 in the upper panel, at 10 uM, Ponatinib demonstrated activity, as predicted by exome sequencing, in both CCA PDOs (7 and 11) with FGFR1 mutations. These finding validated our assays against exome sequencing. However, IC50 tests demonstrated a loss of response between 1 uM and 10 uM (the calculated IC50 were 4.2 and 7.7 uM, respectively). These findings were startling because the maximum concentration (Cmax) that Ponatinib achieves in vivo in patients is 137 nM, which is significantly less than our test predicts is necessary for activity. Extending the duration of drug application past 48 hours is however necessary to validate these findings. Nonetheless, our data argue that direct drug testing on organoids may constitute a useful validation step after exome sequencing and before patient-specific drug selection. The inventors next performed pilot trials with high-content imaging. We utilized DRAQ5 (red) to stain nuclei and YOYO-1 (green in FIG. 9) to stain dead cells. In this experiment, we treated a CCA PDO culture with Gemcitabine, at concentrations from 10 to 0.1 uM. DRAQ5 and YOYO-1 allowed us to follow cell number (red) and cell death (green) in real-time (FIG. 9). Next, in preparation for PDX experiments proposed herein, in collaboration with Dr. Duda (MGH, please see letter) the inventors have established 2 PDX lines from CCA specimens. FIG. 10 is a representative 100X Hematoxylin and Eosin image of a PDX tumor.

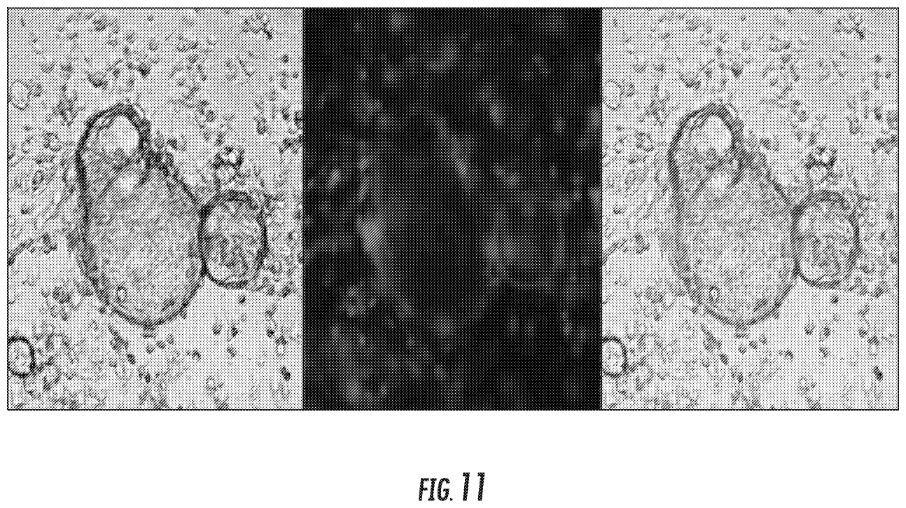

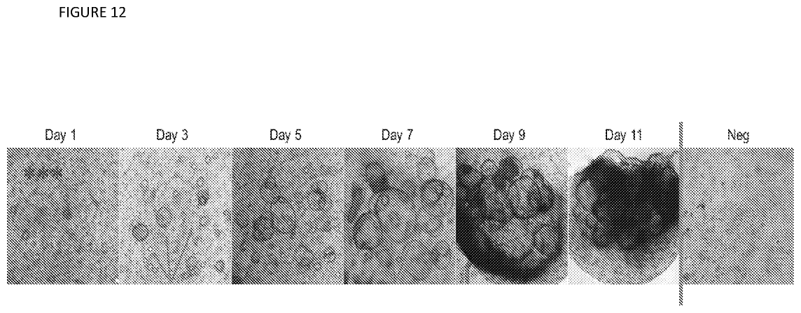

There is mounting evidence that cell types outside of epithelial cancer cells are salient to cancer response to drugs (22). It is not feasible to recapitulate all cell types from a tumor in an ex-vivo model. Here, we will add 2 major types of stromal cells—fibroblasts and endothelial cells—and assess for changes in the PDO positive predictive value in regards to drug efficacy. Future studies can incorporate innate and adaptive immune cells to model response to immune-modulatory drugs. We have prototyped 2 methodologies to create complex PDO (cPDO). First, we directly mixed human CCA PDO (2×10\hat{ }4 cells) with liver stellate/fibroblast cells LX2 (ratio 1:1). LX2 are liver stellate cells immortalized spontaneously by low serum conditions (23). LX2 cells were transduced to express Red Fluorescence Protein (RFP). After 6 days of culturing of CCA PDO mixed with LX2 cells in Matrigel and fed with organoid media adapted by our lab, we noted that both cancer organoids and myofibroblasts appeared healthy as seen in FIG. 11. In follow up experiments, the inventors have used fibroblasts plus endothelial cells (HUVEC) in a 1:1 ratio in direct co-culture experiments. The second model of cPDO (multicellular 3-dimensional organotypic model) was created in collaboration with Dr. Rosangela Mezghanni at University of Maryland (see letter of collaboration and FIG. 12) (24-26). In brief, the inventors utilized 96-well plates to first plate fibroblasts. In brief, the inventors utilized 96-well plates to first plate fibroblasts (CCD-18Co-colon fibroblasts in these early experiments) and endothelial cells (HUVEC) in a ratio of 2×10\hat{ }6:1×10\hat{ }6 per mL of Collagen matrix (prepared with growth factors as described in cited publications). Twenty uL of collagen containing cells were plated per well. After 24 hours, 1,000 cancer cells freshly dissociated from CCA PDO in culture (passage 6) suspended in Matrigel (2.5 uL) were added on top of polymerized collagen I. Media optimized to sustain both the growth of cancer organoids as well as fibroblasts and endothelial cells was added after 20 minutes to allow Matrigel to solidify. Bright field microscopy (2.5×) was performed over the next 14 days as shown in FIG. 12. Note that due to fibroblast-mediated contraction of the matrix, the construct detaches from the well by day 9. The negative control (Neg in the figure) demonstrates stromal cells but no cancer cells.

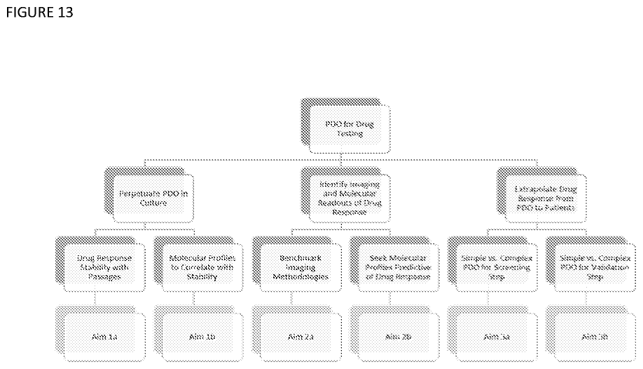

Simple PDOs (epithelial cells only) will be utilized to perpetuate patient-specific cancer cells. Ensuring that PDOs maintained in culture continue to reflect the biology of the original tumor is of paramount importance. These considerations will be evaluated (FIG. 13). Next, the inventors will focus on identifying best methods to assay the effects of drugs on PDOs in culture. These efforts coupled with the identification of molecular markers of drug response will be developed. Last, the inventors plan to identify best PDO model for both the initial drug screening step, as well as for the validation step. Thus, the inventors will evaluate several PDOs for their ability to parallel responses in PDX models.

CCA occurs in males and females. The inventors will enroll a population representing local demographics, including males and females of multiple ethnic and racial categories (see “Inclusion of Women and Minorities”). Based on demographics, the inventors anticipate equal male/female representation, 30-50% white European ancestry, and additional representation from Asian, Hispanic, and African-American ancestry. Sex, age, and ancestry (self-identified and estimated from DNA analysis using Eigenstrat principle components will be investigated as covariates for statistical tests. If necessary, we will analyze individual well-identified ethnic ancestries separately and merge results using standard meta-analysis approaches.

The ability to perpetuate cancer PDO virtually indefinitely creates the need to assess the number of passages up to which the PDO still reflects the drug response/biology of the original tumor. The inventors will perform this authentication, as well as seek DNA/RNA profiles to predict changes in drug responses. Such molecular markers can be used to predict a change in drug response profiles.

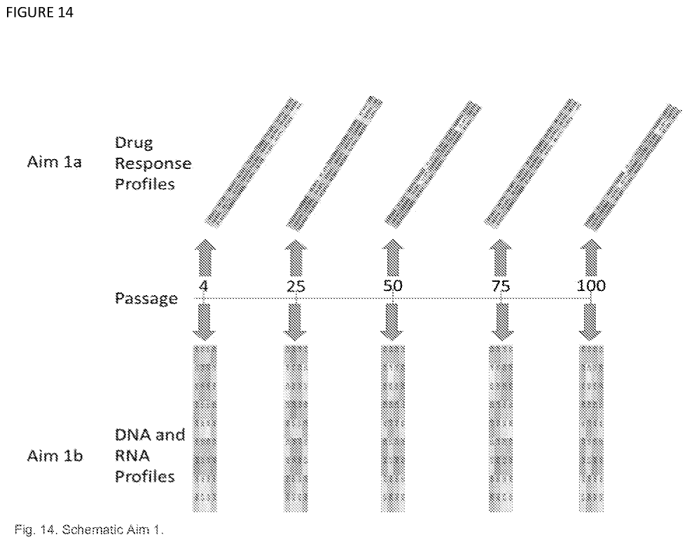

Human cancers accumulate mutations due to genomic instability and selection pressures. While the selection pressures in vitro are different vs. in vivo, they do exist and they can affect drug response profiles. Furthermore, the past utilization of poorly authenticated cancer cell lines has led to reports of unreproducible findings and billions of dollars wasted. The inventors propose to study the effects of passaging CCA PDO on drug response profiles (FIG. 14). Drugs tested on colon cancer organoids 1 month apart demonstrated similar response profiles. To our knowledge, there are no corresponding data for CCA, nor longer duration analyses for PDOs from any organ site. For these experiments, we chose a practical (cost to perform experiments as well as perceived scientific need) cutoff at passage 100. This passage number is approximately 1 year in culture, based on the inventors experience with CCA PDOs. The inventors chose the NCI Approved Oncology Drugs Set VII (129 FDA approved drugs) as a screening library that contains a variety of cancer drug classes. Last, the inventors have performed whole exome sequencing on a fresh cancer tissue from a CCA patient, and on its matched 4th passage PDO. The inventors found no differences in driver mutation profiles (data not shown). Similar data were noted in other cancers. Therefore the PDO genomic profile at passage 4 will serve as a practical proxy for the cancer of origin. The 129 drugs will be tested at 10 uM in triplicate on the 10 CCA PDO already in culture in the Selaru lab at passage 4, 25, 50, 75 and 100, respectively. CellTiter Glo (Promega) will be used, as in preliminary experiments, as the assay readout (example in FIG. 6). PDO/drug combinations that exhibit a response at 10 uM (>50% growth inhibition) will be re-tested at the 5-dose series 10 uM, 1 uM, 100 nM, 10 nM and 1 nM to establish IC50 and Hill coefficient.

There are 2 deliverables that the inventors will achieve. First, once the inventors assign a drug response profile to each passage point (4, 25, 50, 75 and 100) for each of the 10 CCA PDOs (Aim 1a), the inventors will correlate these profiles to DNA/RNA profiles at each of these passage points. The goal is to assess if molecular profiles are indicative, and therefore appropriate, as biomarkers of drug responses. Second, the inventors will compare the stability of DNA and RNA profiles at each of these passage points amongst themselves. The goal is to document the authentication of CCA PDOs as a key biologic resource. To reach these deliverables, for DNA analyses, we will utilize the Illumina HumanCytoSNP-12 v2.1 BeadChip kit for scanning the whole genome for genetic and structural variations (https://www.illumina.com/products/by-type/clinical-research-products/human-cytosnp-12.html). This assay is a panel of ˜300,000 Single Nucleotide Polymorphisms (SNPs) that will identify DNA CNVs (deletions, duplications, amplifications) and copy-neutral loss of heterozygosity. For RNA analyses, we will utilize the Agilent 8X60K SurePrint G3 Expression Microarray, which assays the expression of ˜28,000 unique Entrez genes and ˜30,000 lncRNAs (http://www.genomics.agilent.com/article.jsp?crumbAction=push&pageId=1516). DNA and RNA assays will be performed in the SKCCC Microarray Core.

The inventors will extract DNA and RNA from PDO following standard protocols, as the inventors and others have done. The recommended DNA quantity per sample (200 ng) will be provided to the core facility to run the 8-beadchip workflow. DNA processing will use the recommended workflow of the Illumina BlueFuse CytoChip module including QCs for overall call rate (>98%) and median Log R deviation (<0.2). CNV and LOH status will be extracted from the BlueFuse CGH/LOH reports. For RNA analysis with the Agilent platform, we will use the recommended 10 ng of RNA per sample for the 8X60K, one-color format. RNA data will be analyzed with limma, including methods for normalization, quality testing, and linear models.

The inventors will perform the following 5 types of analyses to integrate drug response/molecular profiles: (a) Trajectory of drug response. The inventors will perform tests of trend (Cochran-Armitage and linear model) for each PDO/drug combination against the null hypothesis that there is no change in IC50 due to number of passages. While mutations that accrue during passaging could conceivably affect IC50 in either direction, the inventors' hypothesis is that rates are low, making multiple changes in different directions less likely. Should the inventors observe many changes in drug response, the inventors can investigate more complex models. The inventors will perform a separate test for each PDO/drug combination. The effect size giving 80% power to detect is R²>0.61 for p<0.05 for a single PDO/drug and R²>0.83 for 0.05 FWER with 1290 PDO/drug combinations. (b) Trajectory of changes to DNA (CNVs) and RNA (gene expression). For each, the inventors will independently test the null hypothesis that the rate of change per passage is constant. For DNA, the inventors will calculate the Bayesian posterior probability for a change in the copy number of each SNP across the genome relative to (i) the baseline copy number; (ii) the copy number at the previous measured passage. The inventors will then sum these probabilities across the genome to calculate the number of CNVs. The inventors will also calculate the number of events using a hidden Markov model to group together CNVs that likely arose from a single amplification or deletion. Next, the inventors will identify time intervals where gene expression changes using a Bayesian change-point algorithm, then sum over the Bayesian posterior probabilities to obtain a global change-point statistic. The inventors will test for correlation between the rate of change of DNA CNV and RNA expression, both overall and locus-by-locus (i.e. CNV at a locus vs. expression levels of transcript from the same locus). The inventors anticipate that most PDO will show a constant, low rate of change of DNA CNVs and of RNA expression, with occasional large bursts corresponding to DNA instability and genomic aberrations. The inventors will determine the number of passages that is 95% likely to not have deviated from simple constant accumulation of changes at the DNA and RNA level for 95% of PDOs. (c) Integration of the drug response data and the DNA/RNA profiling. To test the hypothesis that changes in drug response correspond to changes in genome or gene expression. The inventors will calculate the number of passages 95% likely to avoid changes in drug response for 95% of PDOs. (d) Behavior of known drug targets: many drugs have known targets. For these, the inventors will perform opportunistic analysis to determine whether observed changes in drug response corresponding to genomic or gene expression changes at the target locus. Based on a 0.05 FWER for 129 possible tests, the effect size corresponding to 80% power is R²>0.71 for DNA-based features and responder/non-responder fold-ratio >4.3 or <0.23 for RNA expression assuming typical unit variance on a log 2-scale. (e) Perform the same type of test opportunistically for all loci vs. drugs that display a change in response for at least one PDO. These may identify additional targets or genes in compensating pathways with potential value as targets for combination therapies.

The expected outcomes include: (1) It is possible there will be a change in drug response and DNA/RNA profiles prior to passage 100. These analyses, averaged over 10 specimens, will inform the determination of an approximate/average cutoff for passaging cancer PDOs in cultures. (2) Although unlikely, it is possible that there will be a significant change in DNA/RNA profiles without a significant change in drug response profiles. In this case, the informative cutoffs will be reported in terms of both DNA/RNA and drug response changes. (3) It is possible that we will be able to ascribe a rate limiting step in regards to DNA changes (for example a new driver mutation occurring) that predicts a change in drug response behaviors. Similarly, it is possible to identify an RNA signature that predicts drug response changes (in particular for non-targeted therapies, such as classic chemotherapy agents and/or newer HDAC inhibitors and others). It is also possible that we will identify certain RNA profiles that might predict responses to a certain drug class but not to others (vinca alkaloids vs. targeted therapies).

In one case scenario, the inventors find that there is no drug response/molecular change up to 100 passages. These data will inform future protocols designed to keep the maximum numbers of passages under these numbers. Similarly, a lower passage number identified as a cutoff will similarly serve as guidance. In another scenario, the inventors identify DNA/RNA alterations predictive of drug response changes and these data may serve as dynamic markers of specimen reliability. There is also the possibility that genomic/phenotypic stability may be a function of the initial genomic profile. Last, it is possible that the inventors will not be able to maintain all 10 PDO to passage 100. In this case, the inventors will utilize additional PDOs (from the 30 different CCA PDOs available in the inventor's lab) or establish new PDOs from CCA tumors available through our clinical practice.

Standardized assays are mandatory for clinical-grade tests. The few studies to test drugs on PDO have generally used viability assays, such as the CellTiter-Glo (Promega). The inventors have used CellTiter Glo (FIG. 6) with success, however, the inventors identified few downsides. First, the assay requires fixing cells, making time-course analyses impossible. Second, this assay is an indirect, ATP-based, measure of drug response. The inventors propose label-free image-based assays to quantify growth and invasion. These approaches, if feasible, will be less expensive, more direct and amenable to longitudinal analysis of drug response.

The inventors will implement image analytics for PDO growth as well as to quantify invasion since these cancer phenotypes are the most important clinical determinants of CCA patient survival. Chemotherapy can induce EMT responses and increase the frequency of dissemination and this concern further supports evaluating invasion. Assays that only monitor ATP turnover would fail to distinguish between cytostatic drugs that induce a stationary arrest and those that increase invasion. The inventors have developed imaging pipelines that enable the quantification of invasive and disseminative behavior of cancer cells using non-perturbing white light imaging (differential interference contrast). The inventors have extensive experience in the analysis of invasion and dissemination of organoids derived from murine and human tumors.

For growth assessment, the inventors will measure the area of the organoid in an image. This represents the projected area of organoid volume. In preliminary experiments, tumor organoid area increases linearly with time in Matrigel culture, for at least 96 hours. The inventors will culture and image for 48 hours with vehicle, treat with drug, and then culture and image for an additional 48-96 hours to determine response. Cytostatic drugs will block an increase in organoid area. Cytotoxic drugs will also block an increase in organoid area but may not reduce area. For invasion assessment, the inventors will use spectral analysis of organoid boundaries to convert organoid images into quantitative phenotypes for analysis. In brief, the inventors decompose the boundary into Fourier components, with lower frequency components relating to large-scale deformations from a spherical shape, medium frequency components relating to branching structures, and high frequency components relating to invasive structures. Organoid images will capture 2D cross-sections, which will be traced using an existing interface in ImageJ. Images will be processed through an automated pipeline to extract spectral features. First, the (x,y) coordinates defining the boundary will be interpolated to a parametric curve x(n),y(n), where the index n ranges from 1 to N, with N=1024 to 4096 providing stable results. The individual dimensions will then be Fourier transformed to yield spectral components X(k),Y(k), where the discrete index k ranges from −N/2 to N/2. The spectral power at wave vector k, defined as P(k)=|X(k)|²+|Y(k)|², provides a rotationally invariant representation of the organoid boundary over length scales approximately 1/k relative to the circumference. The component at k=0 represents the overall center of the boundary and is discarded. The k=1 component represents the image size, determined by the number of cells, magnification, and the location of the imaging plane relative to the organoid center; it is used for an overall normalization. The remaining components serve as a signature of the invasiveness of organoid boundary. In addition to rotational invariance, this representation is useful because it is low dimensional, with most power limited to a small number of components, <10 for even the most invasive organoids. The inventors will use the total spectral power summed over modes k=2 and above as a quantitative phenotype for invasiveness. In addition to organoid shape, we will separately track and quantify the number of disseminating clusters.

Each of 30 CCA PDO included here will be screened at 10 uM with the NCI Drugs Set VII (129 FDA approved drugs). The inventors will assess the readout for drug efficacy based on the CellTiter-Glo assay, and the inventors will also perform image acquisition and analysis for growth and invasion. Note in particular that CellTiter Glo and PDO image area both report on cell growth, whereas boundary spectral power reports on invasiveness. These phenotypes may have distinct molecular biomarkers with distinct therapeutic interventions. Drugs that are active at 10 uM (>50% growth inhibition based on CellTiter Glo or PDO volume as estimated from PDO area; >50% reduction in boundary spectral power) will be retested at 1 uM, 100 nM, 10 nM, 1 nM to determine IC50.

The expected outcome is that growth of untreated and treated PDO will be highly correlated when measured by CellTiter Glo and by PDO. Given that CellTiter Glo is an accepted standard for growth assays, strong correlation of PDO area with CellTiter Glo (R²>0.80) will indicate that PDO area is a suitable proxy. For drug response, the inventors will perform two comparisons. First, the inventors will define drugs as active or inactive based on the threshold of 50% growth inhibition (CellTiter Glo) or volume reduction (estimated from PDO area), then determine the PPV of PDO vs. CellTiter Glo as known positives. The inventors will consider 80% PPV as a threshold for use of PPO as a proxy. For compounds active based on CellTiter Glo and PDO area, the inventors will calculate the R² for IC50 values on a log-scale, with R²>0.8 indicating excellent concordance and R²>0.5 indicating a useful substitute assay.

The inventors will compare the list of compounds/IC50s identified through boundary spectral power with those identified through growth-based assays. Compounds that are highly cytotoxic may also reduce boundary spectral power because the PDO will collapse. The inventors anticipate, however, that many compounds that are active in reducing boundary spectral power will have little to no effect on growth. Such compounds may have high utility as anti-metastatic candidates with reduced side-effects from general toxicity.

Cancer genomics has been utilized for (i) the identification of actionable mutations and subsequent (ii) selection of drugs to target that mutation. Here the inventors assess whether DNA/RNA profiles from PDO can provide rigorous, reproducible, and clinically valuable protocols for drug selection. The inventors will directly quantify the effects of 129 drugs on 30 CCA PDO. In parallel, the inventors will seek DNA/RNA profiles to predict the experimentally validated drug efficacy readout. Of note, when compared to DNA/RNA analyses, the ones included here will aid in building predictors. The inventors therefore use more informative data generation. For DNA, the inventors will use exome sequencing of cancer (PDO) vs. normal adjacent tissue, as the inventors have done before for CCA with Agilent SureSelect exome capture, Illumina HiSeq sequencing, and Eland/CASAVA processing. Use of PDO rather than original cancer tissue removes stromal cells, eliminating the need for microdissection. DNA features will be restricted to somatic mutations with likely functional effects: (i) known driver mutations including inferred gain-of-function (GOF) from recurrent missense (oncogenes) and inferred loss-of-function (LOF) from nonsense, frameshift, and deletion mutations; (ii) newly observed candidate drivers defined as recurrent inferred GOF/LOF (>2 samples); (iii) CNVs involving any drivers or candidate drivers. Somatic mutations will be summarized at the gene level as presence/absence of GOF or LOF, and quantitative CNV. The inventors will investigate whether merging GOF and LOF categories will lead to greater power, particularly for LOF inferred from an early stop that may delete a regulatory domain. For RNA, the inventors will use published protocols for organoid-based RNA-seq with Illumina HiSeq followed by a standard BowTie/TopHat/HTSeq/DESeq pipeline.

DNA/RNA features will be tested for association with growth (CellTiter Glo, PDO area) and invasion (PDO boundary spectral power). The inventors will first determine whether sex and other covariates are associated with observed phenotypes (p<0.05); if so, they will be regressed out. DNA tests of GOF/LOF categories will use ANOVA; tests of CNV will use linear model relating phenotypes to CNV status. At genome-wide stringency of 0.05 family-wise error rate and with at most 50 DNA features anticipated based on previous CCA exome sequencing (2), the inventors anticipate that a p-value of 0.001 is appropriate, with 80% power to detect associations with R²>0.39 for 0.05 FWER with 129 drugs. RNA-based features will be analyzed by converting to log-scale and testing linear models for growth and invasion. Assuming unit standard deviation on a log 2-scale and significance threshold of p<1×10⁻⁶ typical for our previous gene expression studies, the inventors will have 80% power to detect transcripts with fold-ratio approximately >2.3 or <0.43 between most responsive and least responsive quartiles. Nominal parametric p-values will be assessed in two ways. First, the inventors will ensure that robust non-parametric equivalents of parametric tests are also significant at a less stringent threshold of 5% FDR. Second, the inventors will perform permutation tests that hold DNA/RNA features constant for each sample while shuffling phenotypes and covariates.

Similarly, the inventors will seek to identify DNA & RNA features that correlate with drug response. We will perform these tests in two ways: (1) significance in an ANOVA model (DNA GOF/LOF categories) or logistic regression model (DNA/CNV and log2-scale RNA) for activity at 10 uM; (2) significance in an ANOVA (GOF/LOF) or linear model (DNA/CNV and log2-scale RNA) for IC50, with inactive compounds assigned a nominal IC50 of 100 uM. Critical R² values for 80% power will depend on the number of samples showing activity. As with pre-treatment phenotypes, we will use non-parametric tests and permutation tests to confirm that findings are robust. The inventors expect the following outcomes: (1) To find that images are superior to chemical-based methodologies. If so, in future studies, the inventors will refine these analyses for complex PDO. If they are not uniformly superior, the inventors will identify applications for which they are best suited. (2) To find molecular profiles (DNA and more so RNA) that are predictive of drug sensitivity of PDO. These signatures will likely be applicable for drug screening, but unlikely to be superior to complex PDO for validation purposes. It is also possible that such profiles can predict the effects of certain but not other drugs. In doing these experiments, the inventors can assign a preliminary role to these approaches as part of a larger strategy to select drugs in a personalized fashion.

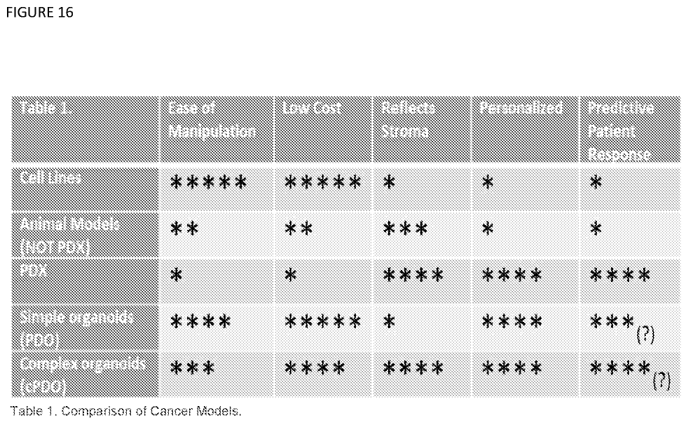

The inventors believe some potential problems & possible solutions are (1) The number of 30 samples is limited by clinical intake and by analysis costs. The inventors will include non-parametric confirmation of robust effects and permutations to confirm p-values. Splitting our samples into discovery-vs-validation cohorts is not appropriate for this sample size, particularly for DNA features where only a small number will have any single somatic mutation. Nevertheless, rigorous thresholds of 0.05 FWER should yield findings that will reproduce in subsequent validation studies. (2) Should power be too low at 0.05 FWER, the inventors will consider relaxing our threshold to 5% FDR also commonly used in such studies. (3) The inventors will use gene-set enrichment analysis, implemented by our own software based on MSigDB categories, to identify possible themes in the candidate DNA and RNA features. The inventors will also use the DrugBank drug-target database to provide support for treatment-response biomarker candidates. All cancer models demonstrate advantages and disadvantages (FIG. 16). Arguably, PDX represent the most accurate models to predict drug activity in a patient-specific manner. PDX are, unfortunately, not yet suited to routine clinical practice due to long engraftment time (up to 10 months, too late for many patients) as well as the high cost associated with testing even several drugs. The inventors will evaluate and adapt patient derived organoid (PDO) models in an effort to replicate the ability of PDX to predict drug responses, at a lower cost, in a shorter time, and with greater clinical applicability. PDX models represent the most accurate system to evaluate patient-specific drug responses. Simple (epithelial only) PDO are the simplest, least expensive PDO, but do not capture the stromal contribution to drug responses). Regarding Complex (epithelial+stromal components) PDO (cPDO), the inventors and others have demonstrated salient roles for the stroma in CCA phenotypes. Similar to pancreatic, prostate and breast cancer, CCAs display a dense fibro-collagenous infiltrate secreted by myofibroblasts. Importantly, myofibroblasts affect CCA response to drugs. Similarly, endothelial cells are linked to drug resistance in cancer patients.

The inventors will utilize the NCI Approved Oncology Drugs Set VII (129 FDA approved drugs) to: 1) contrast PDO vs. cPDO in terms of drug selection sensitivity; 2) benchmark cPDO vs. PDX in terms of drug selection specificity; and 3) retrospectively compare patient response in clinic to PDO/cPDO/PDX predicted responses. The initial screen (relative low cost and good sensitivity) qualifies drug leads for validation. Ideally, due to low cost and ease of manipulation, epithelial-only PDOs will be sufficiently sensitive for screening. The inventors will compare PDOs vs. complex PDO to verify that PDOs are as sensitive as cPDOs for drug selection.

Based on our preliminary experiments, 6 days of co-culture are sufficient to produce matrix contraction and change the expression profiles of CCA cells. In preliminary experiments, the inventors mixed organoids with endothelial media (procured from Lonza) in 1:1 ratio and have shown good viability of all cells up to 10 days after plating. The best model of cPDO in CCA remains to be established (fibroblast source, ratio fibroblasts-cancer cell need to be determined). Fibroblast source may influence response to drugs. LX2 cells are partially activated liver stellate cells immortalized in culture and are the inexpensive, easily obtainable choice. In addition, the inventors will derive myofibroblasts from same human cancer tissue specimens as the inventors utilize to derive cancer organoids. In brief, as the inventors have done in preliminary experiments, 4/5 of the resection piece will be used for isolating cancer cells, allowing 1/5 to be processed separately (collagenase, filtration) and then grown in DMEM/10%FBS/Antibiotics with TGB-β1 supplementation (5 ng/mL), as described previously, to obtain liver myofibroblasts.

The ratio of fibroblast: cancer cells is another important determinant of the level of desmoplasia and consequently response to therapy. We will vary the ratio of fibroblasts to cancer cells from 1:1 (as in our preliminary experiments) to 8:1. In parallel experiments, it was demonstrated a more aggressive cancer behavior in co-cultures where the ratio was 8:1 when compared to 1:1. Absent any data to suggest similarly high impact of percent endothelial cells, as in preliminary experiments, the inventors will keep the ratio cancer cells: endothelial cells 1:1

For each CCA, the inventors will test 1 PDO (simple organoid culture) and 4 cPDOs in regards to their response to each of the 129 drugs in the NCI Approved Oncology Drugs Set VII. As in preliminary experiments, the inventors will use a cutoff of 50% metabolically active cells remaining at 48 hours after applying the drugs as reported by an ATP assay (CellTiter Glo) and create a list of drug leads for each of the 5 PDO models. For 10 CCAs, the inventors will contrast the sensitivity of PDO vs. cPDO in choosing drug leads at this screening stage.

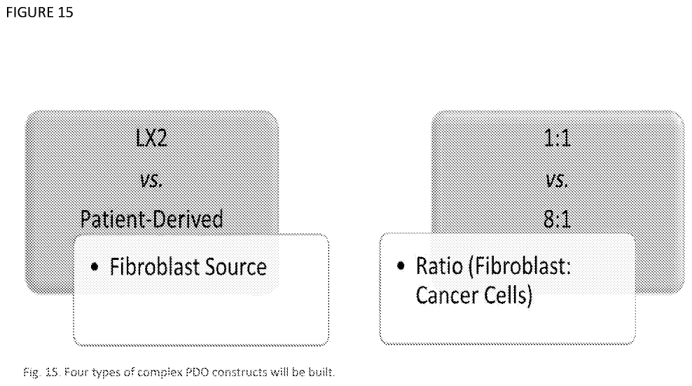

PDX recapitulate drug response profiles seen in patients from whom they were derived. Therefore, the inventors will treat PDX as the best available surrogate of drug response in patients. Recent data suggest that simple PDOs established from breast cancer PDX displayed a positive predictive value of 82.5%. There is no data, however, in CCA or other desmoplastic cancers to report positive predictive value of simple PDO or cPDO. The inventors will test drug leads identified to calculate positive predictive value of both PDO and matched cPDO when compared to drug responses in PDX. First, the inventors will validate the screening results in cPDO-based validation experiments that involve calculation of IC50. The top 5 most effective drugs will be tested at decreasing concentrations (10 uM, 1 uM, 100 nM, 10 nM and 1 nM) in each of the 4 cPDOs (FIG. 15—LX2 vs. patient-derived fibroblasts; equal or high numbers of fibroblasts). The inventors chose the top 5 drugs since in preliminary experiments this was the typical number out of 129 that had an IC50 that was clinically relevant (less than published C Max for that particular drug). The inventors will test these 5 drugs for each CCA cPDO in PDX.
Establishing of PDX. Approximately 10 CCA patients undergo resection at Hopkins every year (please see recruitment table for sex/gender, race and ethnicity). The inventors typically capture all of these patients. Going forward, we will attempt to establish PDX from all of them. CCA is richly desmoplastic, which in our experience diminishes the engraftment rate. Based on data from another desmoplastic cancer, pancreatic cancer, the expected rate of engraftment will be 60-65%. The inventors will perform PDX in NOD/SCID/IL2Rγ-null (NSG) mice since it appears that more immune compromised hosts are more permissive to tumor engraftment. The methodology to create PDX has been extensively described and has been utilized in our laboratory. In brief, CCA tissue obtain from surgical resection will be cut into 2-3 mm 3 pieces in cell culture medium containing antibiotics. Pieces of tissue without necrosis are then selected and placed in Matrigel. Next, two pieces and surrounding Matrigel are implanted subcutaneously, per pocket, in 5-6 week old female NSG mice. Small incisions and pockets are made in the lower back on the left and on the right side, and pieces of tumor are deposited in each pocket. We chose to use subcutaneous implantation (vs. orthotopic) since it allows for close visual monitoring while not presenting with a clear or documented disadvantage. Subcutaneous tumors are harvested when reaching 1,500 mm3. The first generation xenografts (F1) will be cut in 3×3×3 mm pieces and implanted into 5 mice. The rest of the tumor will be flash frozen and stored. As described previously, the next generation of mice (F3) will be treated with drugs. In brief, 3×3×3 pieces of tumor will be implanted in F3 mice. We will implant a total of 50 mice per CCA to account for possible imperfect engraftment (although typically the engraftment rate after the initial generation of mice, F1, is approaching 100%). In our experience, it typically takes between 1 and 6 months for first engraftment, but it can take up to 10 months. The time to grow F2 tumors is less, typically 2-3 months. Once the tumors in F3 mice reaches 200 mm3, 4 mice per group will be randomized to treatment groups.
Drug validation in PDX. For each CCA, the inventors will have selected 5 drug leads (plus a negative control) that will be tested in PDX models at a converted dose matched to human blood exposure, as described previously. The inventors will administer chemotherapy for 21 days, as described. Each of these 6 compounds will be administered to group of 6 tumor-bearing PDX mice. The inventors chose a number of 6 mice per group so that if unexpected mouse death occurs, the inventors still have sufficient numbers for p-value calculations. Thus, the number of PDX mice per CCA specimen is 36. The inventors will validate drug leads in a total of 10 CCA specimens. The total number of mice necessary will be 360. We will measure the size of the tumors in 3 dimensions each other day after starting the treatment and will calculate tumor volume. Reduction in size of tumor will be the primary endpoint. As a secondary endpoint, we will also record survival. Tumor-size response will use a one-side pooled-variance t-test for each treatment group vs. control with a significance threshold of 0.001 accounting for 10 specimens and 5 drug leads tested per specimen. With 5 mice per group, the inventors will have 80% power to detect effects where the reduction in growth is >1.3 of the standard deviation. Survival will be analyzed with standard Kaplan-Meier statistics.
Retrospective correlations with patient responses. First line CCA chemotherapy is typically Gemcitabine+Cisplatin, although other drugs or drug combinations—Capecitabine, Oaliplatin, SFU, FOLFOX (folinic acid, 5FU and oxaliplatin), FOLFIRI (folinic acid, 5FU and Irinotecan), erlotinib, bevacizumab and others—are sometimes utilized. The inventors will retrospectively correlate the patient response in clinic to the response in our models (PDO, cPDO and PDX).
Expected outcomes: (1) Due to microenvironment-dependent resistance mechanisms, the inventors expect to find drugs that are active in PDO but not in cPDO, In this case, to minimize cost and complexity, the inventors will favor PDO for initial screening. However, the inventors should not find drugs that are active in cPDO but not active in PDO. If the inventors do, this result would motivate screening in cPDO models instead of PDOs. (2) A recent study showed that simple breast cancer PDOs had a positive predictive value of 82.5%. Note that these cancers were not selected for high fibrous content (which is the case with virtually every CCA). In addition, the PDO were established from PDX, not directly from patients, which may account for the high concordance. The inventors expect that for CCA, cPDO will offer increased specificity vs. simple organoids in regards to drug selection. (3) The inventors expect that the presence and numbers of fibroblasts in CCA cPDOs will affect drug responses since increasing numbers of fibroblasts in co-culture with CCA increased the cancer aggressiveness cancer. The inventors do not expect that the source of fibroblasts (cell line vs. patient-matched liver myofibroblasts) will make a significant difference.
Potential problems & possible solutions: (1) CCA is a desmoplastic cancer that, in the inventors hands, has engraftment rates of approximately 65%, similar to pancreatic cancer. The inventors will collect an additional number of 30 samples over the next 3 years (in addition to the 10 the inventors already collected), the inventors expect to generate the required PDX numbers. (2) It is possible that patient-derived myofibroblasts will demonstrate higher utility (increase drug selection PPV) vs. a liver fibroblast cell line (LX2). Since for some patients the tissue may be insufficient to derive both cancer organoids and fibroblasts, the inventors will purchase primary human stellate cells from Lonza/Triangle Research Labs and contrast with patient-matched liver fibroblasts in regards to drug responses. (3) If neither simple nor cPDO accurately predict the response in PDX, the inventors will implement, as in preliminary experiments, another cPDO construct. The inventors will construct cPDOs by adding cancer cells mixed with Matrigel to stromal cells already embedded in a collagen I matrix. (4) In case the inventors cannot match PDX PPV with our cPDO models, the inventors will test other assays/readouts. (5) The inventors' threshold of p<0.001 is highly stringent. Should effects be significant at p<0.05 but not at p<0.001, the inventors will calculate effect sizes. If effects appear clinically relevant, the inventors will determine whether increasing group sizes up to 12 animals per group will achieve sufficient power.
   Establishment of Multiple PDO Lines from Geographically Distinct Regions of Human Liver Cancers

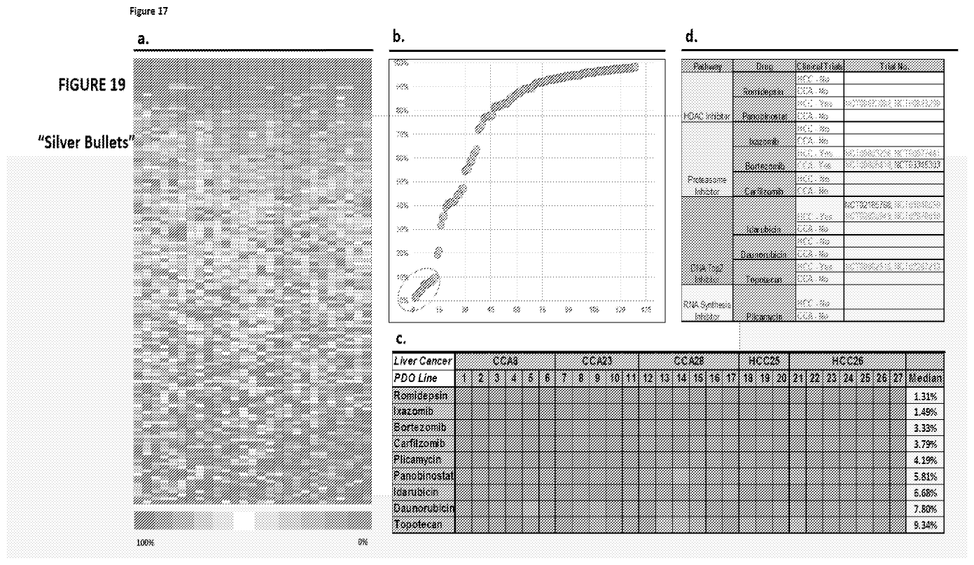

Patient Derived Xenografts (PDX) models can be used to recapitulate cancer features and behaviors as well as for anti-cancer drug screening, either to characterize activity of new lead compounds, or in a highly personalized fashion to identify best chemotherapy for a specific patient. As described in, as well as in the inventor's experience, there is a wide variability of human tumor engraftment in immunocompromised mice. Although data regarding engraftment of CCA samples in immunocompromised mice is sparse, the inventor's experience (data unpublished) mirrors the poor rate (less than 6%) published recently in one study. Engraftment of HCC is also problematic. Recently, PDOs were successfully established from primary liver cancers and their histology, genetic and transcriptomic profiles were shown to mirror their corresponding primary human tumors. In a similar fashion, the inventors established PDO cultures from a human intrahepatic CCA. A surgical resection specimen from a CCA as well as matched histologically matched liver were obtained from a patient with an intrahepatic CCA. In line with the inventor's focus on biologic replicates obtained from same cancer tissue, the inventors cut the resection piece into 20 tissue slices as shown in FIG. 17A. Tissue was minced, washed, subjected to single cell separation and plating embedded in growth factor reduced Matrigel, according to published protocols. As shown in FIG. 17B, we noted an epithelial organoid appearance of cystic growth starting with Day 2 after plating. The cystic structure (the PDO) continued to grow to day 10, as shown, at which point the PDO culture was split. Representative images for this PDO culture are shown at passage 3 (P3 in FIG. 17B) as well as passage 6 (P6 in FIG. 17B). The inventor's utilized the tissues slices for histology, tissue banking and other analyses, and have we established PDO lines from 6 segments.

To confirm that the cystic PDO structure identified in white light images was indeed derived from a CCA, we embedded patient CCA tissue, as well as matched PDOs extracted from these CCA tissues, into optimum cutting temperature (OCT) compound, cut slides, and stained with representative antibodies. FIG. 17C (primary tumor) and FIG. 17D (matched PDO) illustrates positive staining for the epithelial marker EPCAM, bile duct marker cytokeratin 19 (CK19), and stem cell markers LGR5 and SOX9. The inventor's utilized CK19 since this marker is a staple of immunohistochemistry/immunofluorescence for hepatobiliary tumors. CK19 was historically utilized to differentiate hepatocellular cancer (HCC) from CCA but in later years it was found that CK19 could also be expressed in a small subset of aggressive HCCs. To further demonstrate that the PDOs are indeed derived from CCA tissue, the inventors performed hematoxylin and eosin (H & E staining, FIGS. 17E and 17F) and immunohistochemistry with another bile duct marker (cytokeratin 7—CK7) and with a marker staining mucin produced by bile duct epithelial cells (mucicarmine). CK7 is routinely utilized in the clinical diagnosis of CCA, since CCAs tend to express this marker, while colon cancer metastatic to the liver does not. The surgical human CCA specimen expressed CK7, as did the PDO established from this CCA specimen (FIGS. 17E and 17F). Mucin production by a subset of CCAs has been noted and it appears to impact clinical behavior as well as response to therapy in a preliminary ex-vivo study. We noted mucin production by the CCA specimen as well as by the matched PDO (FIGS. 17E and 17F).

A human primary HCC specimens was processed similarly to the CCA specimen. In brief, 7 geographically distinct pieces from the same surgical specimen were utilized to establish 7 corresponding sister HCC PDO lines. The inventors confirmed that PDO cultures are similar to primary cancer.

### Drug Testing and Effect Quantification on a PDO Line

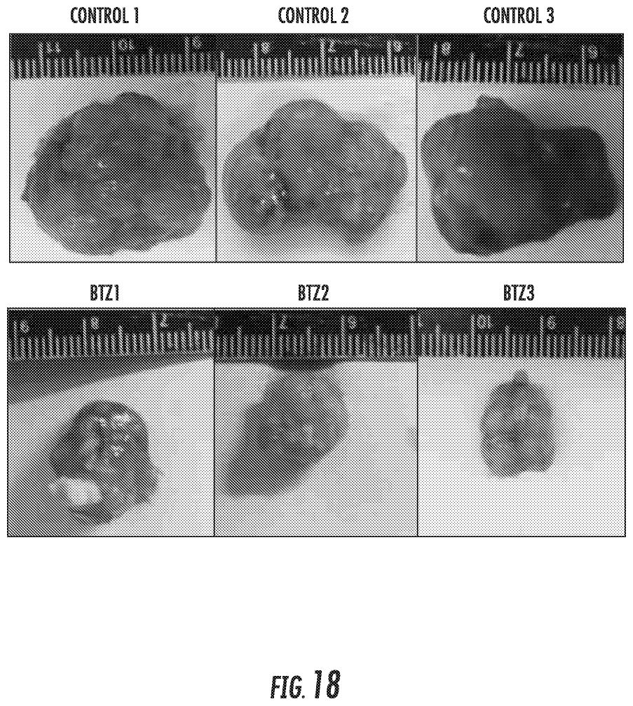

Initial drug testing experiments were performed on one PDO line, with a 9-drug library, at a standard HT screening drug concentration of 10 uM. For these experiments, we utilized a CCA PDO line. For positive control, we used 10% Triton X, while for negative control we used DMSO a25 same concentration used to dissolve drugs. For these experiments, we utilized white light images and equated the killing effect of drugs into shrinking of PDO cystic structures. As shown in FIG. 18A, we were able to appreciate differential killing power based on the shape of the cystic PDO structures. Of note, we also found that Cisplatin (a first line CCA drug) did not appear to have any effect on the number or size of the cystic PDO structures. In addition, Gemcitabine (the other commonly used first line CCA drug) appeared to have a moderate cytostatic but not a cytocydal effect. Of note, this patient was treated with Gemcitabine and Cisplatin with a modest response. Interestingly, Vinorelbine appeared to be effective (FIG. 18A). To our knowledge, there has been a single case report to mention successful utilization of a drug cocktail that included Vinorelbine in a CCA patient. Next, we expanded the drug panel to 137 drugs tested on the same PDO clone. Of these drugs, 8 are currently in development and 129 drugs were provided by The NCI Approved Oncology Drugs Set VII (129 FDA approved cancer drugs, please find more information at https://wiki.nci.nih.gov/display/NCIDTPdata/Compound+Sets). Interestingly, we found that bortezomib, a first generation proteasome inhibitor, exhibited excellent cancer inhibitory effects (see FIG. 18B for a comparison between Cisplatin and Gemcitabine—first line therapy choices in CCA and Bortezomib). Although a phase II trial with bortezomib in advanced biliary tract cancer as a single agent did not result in objective responses, the rate of stable disease was improved, as was the time to progression. Our data suggests that, at least in some patients, bortezomib may be a reasonable chemotherapy choice. Next, we asked if combination therapy (as it is standard in clinical practice) has a different effect on a CCA PDO vs. single agents. To this end, we tested the 5 chemotherapy agents typically used in CCA patients, as well as several combinations (FIG. 18C). We noted that the combination therapies tended to mirror the effects of the single agents without any additive effects. Next, we sought a fast, reproducible and relatively inexpensive read out of cell death (or inhibited proliferation). We chose CellTiter-Glo (Promega), which is an ATP-based cytotoxicity assay that quantifies metabolically active cells. We have determined that there was good correlation between direct white light image analysis (as in FIGS. 18A, 18B and 18C) and readouts from CellTiter-Glo (data not shown) and also measured the the Z-factor to be 0.84 which is excellent for HT drug screening. Therefore, we have used CellTiter-Glo for all subsequent experiments. FIG. 18D illustrates a typical, color coded, viability/CellTiter-Glo readout from running the 137 drug library on same CCA PDO. Note that the vast majority of drugs have little to no impact on viability (blue colored boxes) while 9 drugs (6.5%) demonstrated excellent killing activity (less than 5% metabolically active cells). By using CellTiter-Glo, we quantified the viability of cells treated with single agents as well as combinations (as in FIG. 18C). As FIG. 18E demonstrates, the effect of combination therapy appears driven by the more efficacious of the 2 drugs of the pair. For the top active drugs, we performed drug testing at 10 uM, 1 uM, 100 nM, 10 nM and 1 nM to derive inhibitory concentration (IC) 50 curves. FIG. 18F demonstrates a representative IC50 curve for Gemcitabine.

### The Identification of “Silver Bullets” by High Throughput Drug Screening in a Large Cohort of Liver Cancer PDO Lines

To investigate if liver cancer sister PDO lines can be utilized for high-throughput drug screening, we collected two additional human primary CCA surgical specimens and an additional human primary HCC surgical specimen. From each cancer specimen, we established between 3 and 7 PDO lines (Table 1).

TABLE 1
Summary of human primary liver cancer specimens, demographics
and laboratory derivation of sister PDO lines.

| Lab | | | | | | PDO |
|---|---|---|---|---|---|---|
| ID | Disease | Age | Gender | Race | Predisposing | Lines |
| CCA8 | iCCA | 63 | female | hispanic | None | 6 |
| CCA23 | iCCA | 60 | female | white | None | 5 |
| CCA28 | iCCA | 57 | female | white | None | 6 |
| HCC25 | HCC | 71 | male | white | HCV | 3 |
| HCC26 | HCC | 69 | female | white | HCV | 7 |
| Total | | | | | | 27 |

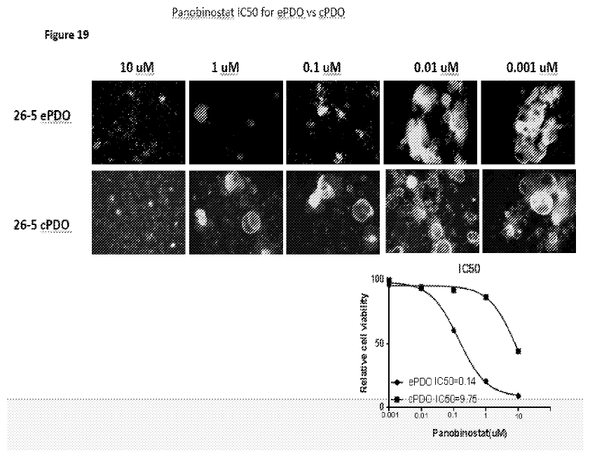

Next, each PDO line from each cancer was subjected to treatment with the NCI VII 129-drug panel and the killing effect was measured with CellTiter-Glo (measuring percent viability 4 days after treatment was applied). The experiment produced 27 readings (from 0% to 100%, one for each liver cancer PDO line) for each of the 129 drugs, for a total of 3,483 data cells. These data is shown in a color coded fashion in FIG. 19A. The inventors noted that there are a number of drugs that appear to be uniformly effective across all 27 liver cancers PDO lines (red data cells at the top of the heat map in FIG. 19A). FIG. 19B displays the median survival across all 27 liver cancer PDOs. Note that there are 13 drugs (circled with red in FIG. 19B) that display uniform killing capacity across all 27 liver cancer PDO lines, manifested through a median survival of less than 10% across all lines (“Silver Bullets”). However, upon further inspection of these 13 Silver Bullet drugs, the inventors found that Omacetaxine, Dactinomycin, Doxorubicin and Mitomycin each had at least 1 PDO line where they were ineffective. Therefore, we reduced the list of Silver Bullets that are effective across all 27 liver cancer PDO lines to 9 drugs (FIG. 19C). Interestingly, we noted that these 9 Silver Bullet drugs belong to only 4 classes of anti-neoplastic agents (histone deacetylase—HDAC, proteasome inhibitors, DNA Topoisomerase II inhibitors and RNA synthesis inhibitors—FIG. 19D). Of note, there was no targeted therapy (tyrosine kinase inhibitor) agent included in this group. A search of completed or ongoing clinical trials at clinicaltrials.gov demonstrated that only 2 out of these 9 Silver Bullet drugs had ever been tested as systemic chemotherapy in liver cancers (panobinostat and bortezomib, FIG. 19D). Another 2 agents (idarubicin and topotecan) had been tested as topical therapy (trans arterial chemo embolization—TACE—with beads). Also of note, only 1 of these drugs has ever been tested in a clinical trial in CCA (Bortezomib). Out of these trials, only 2 are still recruiting.

### Inter Patient Functional Heterogeneity—The Identification of Inter-Patient “Divergents”

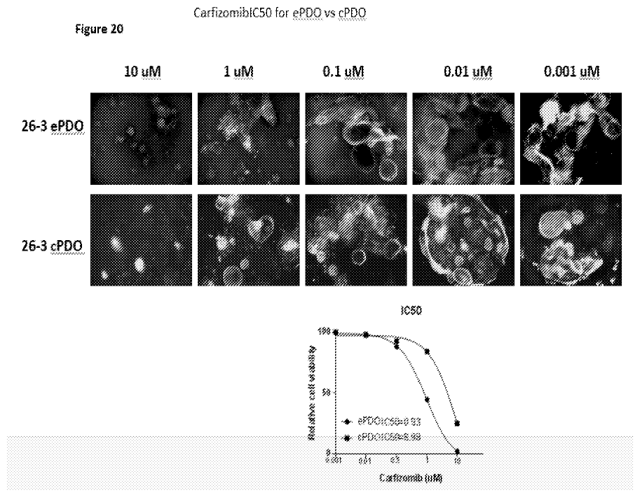

The drug testing results indicates that a number of drugs might be efficacious in a subgroup of patients. For example, dasatinib, previously reported as efficacious in a liver cancer organoid drug testing study, appears to work well in HCC26, but not at all in HCC25 or in CCA8. To pick out inter patient divergent drugs (i.e., drugs with discordant effect across the 5 patients), we calculated the mean cell viability per patient cancer specimen by averaging the effect of the drugs across all PDO lines established for the same patient. This mean cell viability per patient is a measure of how the tumor, as a whole, responds to a certain drug. Next, we calculated the standard deviation of the 5 means (one per patient) and ordered the data in decreasing order of the standard deviation. We excluded drugs that did not induce a significant cell killing in at least one of the 5 cancers (arbitrarily defined as 30% viability or less in at least one specimen). The top interpatient divergent drugs are shown in FIG. 20A. Note that while some inter patient divergent drugs work well in just 1 cancer (such as dactinomycin or vincristine), others work in majority of cancers, but not in all of them (such as gemcitabine or sorafenib, or doxorubicin). Furthermore, it is worth mentioning that these 3 drugs (gemcitabine, sorafenib and doxorubicin) are in clinical use for CCA (gemcitabine) and HCC, respectively (sorafenib and doxorubicin). The fact that some patients appear to respond, while others don't, suggests that PDOs from human liver cancers may play a role as a clinical decision making tool. To further visualize the inter-patient divergence of drug effects, the inventors constructed a heat map of drug responses per each patient (utilizing the mean cell viability over all clones from each patient, FIG. 20B). Divergent drugs can be found based on the diverging color in a subgroup of samples vs. the rest of the samples.

### Intra Tumor Functional Heterogeneity—The Identification of Intra-Tumor “Divergents”

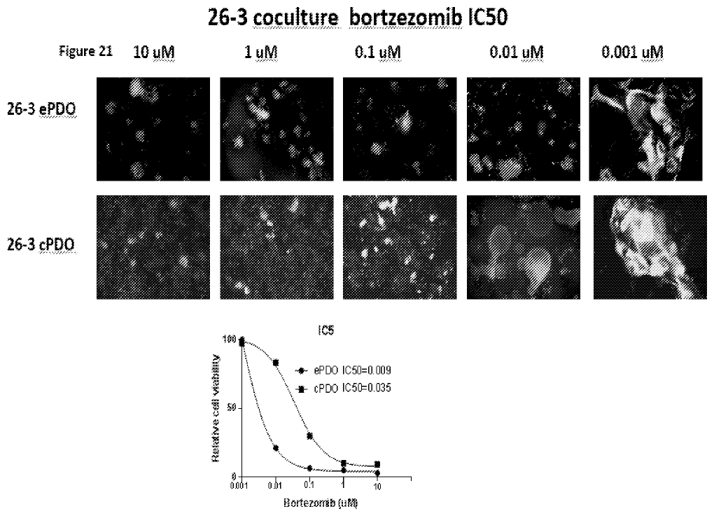

The results allows the visual identification of drugs that have a divergent effect among PDO lines established from a single patient tumor. This intra tumor functional heterogeneity was investigated by calculating the standard deviation of a drug response across all PDO lines established from a patient tumor. FIG. 20C displays a heatmap of patient cancer samples (x-axis) and drugs (y-axis). The yellow bars identify, for each patient, those drugs that display a high standard deviation across the intra-tumor PDO lines. Of note, targeted drugs (tyrosine kinase inhibitors), such as ceritinib, sorafenib, dasatinib and others were well represented as intra-tumor divergent drugs. The inventors chose one of the cancer specimens (CCAS) for further in-depth studies of intra-tumor functional heterogeneity and the identification of its drivers. The inventors noted that several drugs displayed a fairly uniform killing effect over these 6 PDO lines, while others had substantially differing effects. To visualize this phenomenon, we calculated the standard deviation of each drug effect across the 6 PDOs (FIG. 20D). As seen in the graph, most drugs had a similar effect over the 6 PDO lines, as evinced by the low standard deviation (left-most, horizontal side of the curve in the figure, from drug 1 to approximately drug 113). Of note, drugs that displayed low standard deviation in terms of effects on cancer cells are either equally effective, or equally ineffective. For example Ixazomib, Carfilzomib, Romidepsin, Plicamycin, Bortezomib, Mitomcyin and Idarubicin were each effective across the 6 PDO lines (FIG. 20E). At the other end of the spectrum, Thalidomide, Gefitinib, Letrozole, Idelalisib, Melphalan, Lapatinib and Cisplatin were uniformly ineffective in each of the 6 sister PDO lines (FIG. 20E). The drugs displayed at the right of the graph in FIG. 20D (vertical side of the curve) displayed significant variation in terms of killing effect over the 6 PDO lines (FIG. 20F). For example, Belinostat demonstrated moderate activity in 1/6 PDO lines and generally poor activity in 5/6. Dasatinib worked well in 3/6, had moderate activity in 2/6 and poor activity in 1/6 PDO lines. Interestingly and of high clinical interest, Gemcitabine showed moderate activity in 1/6 and poor activity in 5/6 PDO lines. Some drugs, such as Ceritinib demonstrated a sharp difference between high activity in 3/6 and almost no activity in the rest 3/6 PDO lines. A principal components analysis by drug effectiveness across PDO lines further demonstrated a central cluster that is populated by drugs which had similar effects across all 6 PDO's (FIG. 19D). It is logical to hypothesize that drugs that showed differential effectiveness across the 6 PDOs are likely not exerting their effects through a general, non-discriminatory dose-related mechanism. By extension, it appears likely that these drugs that show differential killing power across sister PDO lines established from same patient cancer, are biologically important and that their effects are reflective of functional heterogeneity across those 6 PDOs. Next, we evaluated the potential impact of drug mechanism of action and by extension drug class, onto observed effect across the 6 sister CCA PDO lines. To this end, we first chose drugs that induced 30% or less viability in at least one of the 6 sister CCA PDO lines when tested at a 10 uM concentration. This arbitrary cutoff was chosen based on our experience that a drug that induces a modest decrease in cancer cell viability (30% or more) at a concentration of 10 uM, loses its effects at lower, more physiologic doses (typically 1 uM and less). Based on this cutoff, we chose the top 27 out of the total of 129 drugs (Supplem FIG. 21B). We found that proteasome inhibitors appear to be effective across all 6 sister CCA PDO lines. Of note, there is no known genetic alteration (mutational or otherwise) to predict the response of human cancers to proteasome inhibitors. Furthermore, although early efforts to identify transcriptomic biomarkers of sensitivity to proteasome inhibitors are encouraging, they had not been clinically validated either. According to (Supplem FIG. 21B, histone deacetylase (HDAC) inhibitors also appear to be fairly effective across all 6 sister CCA PDO lines, which is not surprising giving exome sequencing studies that identified chromatin modifying genes as top mutated genes in CCA. Similar to proteasome inhibitors, however, there are no genetic tests that can predict response to HDAC inhibitors. In addition, exome or whole genome sequencing could not predict response to microtubule inhibitors, DNA topoisomerase 2 inhibitors or the “Others” class in same (Supplem FIG. 21B, since the mechanism of action for these drugs is generally not DNA mutation-specific. Of interest, we notice a wide response to these non-targeted drugs among sister CCA PDO lines (examples include Epirubicin as well as Omacetaxine), suggesting that functional heterogeneity (i.e., response to chemotherapy) may be clinically relevant, and conceptually impossible to be predicted by exome/genome sequencing or other indirect, biomarker based techniques. By extension, it appears that multiple sampling of human CCAs may be necessary to provide with an in depth and potentially clinically relevant image of chemotherapy response.

### Neither Exome nor Transcriptome Sequencing Analyses can Accurately Predict Response to Therapy

A principal component analysis that evaluated inter-PDO line similarity as a function of each line response to treatment demonstrated that, based on their functional heterogeneity (i.e., response to a panel of 129 cancer drugs), the 6 sister PDO lines appear to cluster in 3 distinct groups (FIG. 21A). CCA8-7 and CCA8-11 appear similar to each other, as do CCA8-9, CCA8-10 and CCA8-5 amongst themselves, while CCA8-6 does not appear similar to any of the other CCA PDO lines (FIG. 21A). In order to determine whether the functional, drug response intratumor heterogeneity was driven and potentially predictable by underlying variation at the DNA or RNA level, we performed whole exome as well as RNA sequencing on these 6 sister CCA PDOs. We posited that if DNA or RNA profiles are found to correctly identify functional, drug-response groups, then functional testing of human cancer specimens is not necessary. Biopsying protocols followed by molecular analyses, then, should be sufficient to correctly predict the personalized drug choice for a cancer patient. Coverage of the targeted regions was good, with average 141× depth sequencing, and 98% of the exome sequenced to at least 20× depth. While data in human CCA lacks, genetic intratumor heterogeneity has been recognized in other human cancers, such as renal cell carcinoma and HCC. To investigate if genetic intratumor heterogeneity exists in human CCA, we built a phylogenetic tree based upon somatic protein-coding mutations (FIG. 21B). We found little divergence between four sister CCA PDO lines (CCA8-5, CCA8-7, CCA8-9, and CCA8-11), indicating that they were likely all derived from a single clonal population. The other 2 sister CCA PDO lines (CCA8-6 and CCA8-10) were more genetically divergent, both in relation to each other, as well as to the group of the other four sister CCA PDO lines, indicating a separate population represented by each of the two PDOs. In addition, we noted that this classification of sister CCA PDOs that is based on genetic similarity/variance does not overlap with the groups identified functionally (FIG. 21A vs. FIG. 21B). Next, to evaluate overall relationships between the 6 sister CCA PDO lines at the level of gene expression, we analyzed RNA sequencing data collected from each of PDO lines. Using PCA of the fragments per kilobase per million mapped reads (FPKM) for each transcript in the NCBI human reference sequence database, the inventors determined that the distribution of PDO's along the first and second principal components showed widely distributed heterogeneity, even among the 4 PDO lines that displayed genetic similarity in the exomic mutation analysis above. Of note, CCA8-5, CCA8-7 and CCA8-9 are very similar in regards to their exome profile (FIG. 21B) but they are each part of a different group in terms of their transcriptomic profiles (FIG. 21C). This discordance likely reflects a number of transcription modulators that have previously been implicated in tumor heterogeneity, including epigenetic factors and microRNAs. In order to attempt to link observed heterogeneity in drug response to variation in either DNA mutation or gene expression, we first performed a global assessment of drug response heterogeneity using PCA. This found a relatively diffuse distribution in principal components 1 and 2 that did not correspond to that of the RNA PCA. On a more granular level, we tested for pairwise association between individual DNA variants or gene expression levels and drug responses, across the 6 PDO's, but this did not identify any statistically significant associated pairs, after correcting for multiple testing.

The American Society of Clinical Oncology (ASCO) affirms the need to develop standardized assays for direct drug testing in patient-derived cancer samples. The promise of precision oncology has been, to date, centered mainly on genomics. The paradigm is attractive: identify an actionable mutation, then choose the drug that targets that mutation. One of the caveats, however, is the fact that, employing existing drugs and protocols, only 2-6% of cancer patients are successfully paired with a drug. Therefore, the need to develop direct, accurate, replicable, quick and inexpensive functional drug testing on patient-derived cancer samples is urgent.

Drug testing on patient-derived specimens requires cancer models that recapitulate the biology and predict the response of the cancer of origin. To date, several systems had been employed, including cell lines (in vitro and xenotransplanted in immunocompromised animals), genetic animal models, patient derived xenografts (PDX) and recently patient derived organoids (PDO). Arguably, PDX models represent the gold standard in recapitulating cancer biology. Importantly, PDX was found to have in excess of 80% drug response positive predictive value in patients. There are, unfortunately, practical limitations to using PDX routinely to inform drug choice in oncology practice. PDX models are expensive. They are also slow (it may take up to 10 months to 1 year to establish some of these models). In this study, the inventors benchmark and standardize PDOs with the purpose of achieving high accuracy (similar or better than PDX) in a reproducible, inexpensive and quick fashion. Data we accrued to date shows that we are able to (i) successfully establish PDO from all CCA samples collected to date; (ii) develop and adapt protocols to keep these patient-specific cancer samples in culture virtually indefinitely; (iii) develop and adapt protocols to test up to 1,500 drugs in each patient derived culture (high-throughput); (iv) incorporate stromal cells to better recapitulate the biology and drug response of CCA; (v) perform initial drug screening as well as validation testing and inhibitory concentration 50 (IC50) calculations; (vi) provide with a drug sensitivity report in 6 weeks or less since specimen collection from patients.

The inventors identified patient-specific patterns of intra-tumor heterogeneity, implying that lessons about variable drug response for one individual's cancer cannot be applied to another individual's clinical scenario without additional patient-specific experimentation.

To the best of the inventors' knowledge, few prior studies have directly and simultaneously evaluated intra-tumor heterogeneity at the level of somatic mutation and that of variable gene expression. The present invention is the first to demonstrate that, on a global scale, a genetic clonal relationship across tumor tissue samples does not necessarily imply increased similarity in their gene expression profiles. Multiple possible mechanisms that affect differential gene expression in cancer, independent of DNA mutation effects, have been proposed and investigated, including DNA methylation [PMID 28423542], epigenetic histone modification [PMID 16369569], and micro RNA expression [PMID 26681654, PMID 48922647].

As the inventors evaluated the relationship between somatic, exomic mutations and gene expression, they also sought to analogously interrogate the level of similarity between sequencing-based heterogeneity and variable drug response of PDO's. The inventors were unable to relate, on a global level, either exomic genetic variation or gene expression to overall drug response profile. This was not especially surprising, as studies to date have not identified widespread patterns in the relationship between such pharmacogenomics biomarkers and clinically applicable drug efficacy [PMID 25338550]. Numerous factors on the tumor, individual, and population levels contribute to complex biology underlying a cancer's susceptibility to various anti-cancer agents. Our efforts to identify specific associations between particular mutations or genes and drug response profiles were limited by the small number of PDO's used in this study. However, in the clinical setting, for technical and cost reasons, only a limited number of PDO's can be generated to inform an individual patient's treatment; therefore, the constraint imposed by limited sample size is unlikely to be overcome. Therefore, performing assays of indirect biomarkers, such as DNA or RNA profiling, would seem to have much lower yield in clinical application, compared to direct interrogation of a tumor's heterogeneous response profile to a panel of potential drugs.

The patterns that emerged when we assessed intra-tumor and inter-tumor heterogeneity of drug response profile hold implications for how the results of PDO drug screening can be applied to individualized treatment in the clinical setting. The relatively similar drug response profiles for each cancer, when averaged across PDO's to eliminate intra-tumor heterogeneity, imply that individualized therapy is unlikely to result from assessing for inter-individual variability alone, as would be conducted using single biopsy specimens. While the presence of high cancer cell killing for some drugs across all cancer samples would seem to indicate that those drugs stand to be used universally to treat cholangiocarcinoma, this in fact more likely reflects in vitro conditions that favor the cytotoxicity of those specific medications.

In contrast to inter-tumor relative uniformity, the presence of some intra-tumor heterogeneous drug responses indicates that the use of PDO's in an in vitro assay provides biological, and also potentially clinical, value for at least a subset of drugs in the screening panel. Specifically, resistance to a drug that is localized to only a portion of a tumor implies a resistance factor that has emerged during the development of that tumor, and that needs to be overcome by treatment with another drug. The underlying reason for the difference between intra- and inter-tumor drug profile comparisons may lie in the accumulated genetic and non-genetic factors that confer drug resistance to cancer cells and lead to intra-tumor variability, in contrast to the root, or shared, somatic mutations that cause cancer to arise. Therefore, targeting a drug (or likely a combination of drugs) to the localized susceptibility patterns of a particular tumor may lead to improved efficacy of cancer cell killing.

Current personalized clinical oncology approaches include biopsying of the tumor followed by exome analyses to identify “actionable” mutation(s). The NCI-Match trial showed, however, that only XX % of cancer patients who underwent this approach to date as part of the trial were successfully matched with a targetable drug (CITATION). This fact suggests that the frequency of actionable mutations may not be sufficiently high to allow this exome-sequencing that informs drug choice approach to significantly impact clinical cancer patient care. Therefore, it appears that exome sequencing as a patient individualized data interrogation methodology to inform drug treatment may not be clinically impactful. In addition, the NCI-Match trial has shown that even those patients for whom exome sequencing did identify an actionable mutation did not survive significantly longer (CITATION). Additional discreet reports exist of targeted therapy that initially demonstrates good response, only to fail later due to either selection or emergence of a resistant cancer clone (CITATION NEJM melanoma in young person case). The reasons for which patients who do harbor actionable mutation(s) and who are successfully paired with a targeted therapy fail to have a significantly better clinical outcome are not clear. Data presented here argues that one potential explanation lies in the intra-tumor functional heterogeneity, which may impact clinical outcomes more than the presence of an actionable mutation in one of the clonal branches of the tumor. In addition, data presented here further argues that neither genetic heterogeneity, nor expression heterogeneity are able to accurately predict functional, drug-response intra-tumor heterogeneity.

Patient derived organoid (PDO) models of human cancers are utilized increasingly for research purposes. In addition, PDOs may appear as good candidates to augment or replace exome NGS (next generation sequencing) methodologies to inform best drug choice in a patient-specific fashion. Our data suggests that intra-tumor genetic heterogeneity does not mirror the expression heterogeneity and neither of them predict the drug response, functional, heterogeneity. However, since the drug response heterogeneity is the clinically important variable, these data cast a shadow of doubt onto clinical utility of genetic profiling of tumors for informing clinical drug choice. In addition, these data bring center stage the concept of multiple cancer biopsying protocols to ensure that the functional intra-tumor heterogeneity is well represented a PDO-driven methodology to inform patient-specific drug choice.

### EXAMPLES/METHODS

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

### Example 1

### Media Compositions for Growing Liver Cancer Organoids/Mini-Cancers

A) Basic Media For Making Mini-Cancers:
Advanced DMEM/F12 (Gibco—Ref: 12634-010). Add 1M HEPES buffer (Quality Biological) AND GlutaMAX (Gibco Ref: 35050). For 500 mL DMEM/F12 you add 5 mL HEPES and 5 mL GlutaMAX. Add Puromocin 1 mL Basic media can be used for up to 1 month

B) Growth Media for Establishing Mini-Cancers and for Passaging Mini-Cancers:

| | | | Cat |
|---|---|---|---|
| Basic Media | 7 | mL | As above |
| HGF | 10 uL | (F Conc: 25 ng/uL) | Peprotech (cat: 100-39H) |
| FSK | 10 uL | (10 uM) | TOCRIS(cat: 1099) |
| FGF10 | 10 uL | (100 ng/mL) | Peprotech(cat: 100-26) |
| Gastrin I | 2 uL | (10 nM) | Sigma (10047-33-3) |
| NAC | 40 uL | (1.25 mM) | Sigma (A7250) |
| B27 supplement | 400 | uL | Gibco Ref: 12587-010 (50X) |
| N2 | 800 | uL | Gibco Ref: 17502-048 (100X) |
| Nicotinamide | 400 uL | (10 mM) | Sigma (N3376) |
| Wnt3A-conditioned medium | 6 mL | (30%) | Made in the lab - 293T |
| | | | cells (purchased from |
| | | | ATCC) |
| R-spondin1-conditioned | 4 mL | (20%) | Made in the lab - 293T |
| medium | | | cells (purchased from |
| | | | Trevigen) - HA-R- |
| | | | Spondin1-Fc293T) |
| EGF | 2 uL | (50 ng ml⁻¹) | Sigma(62253-63-8) |
| Noggin | 100 ng/mL | (2 uL) | Peprotech |
| A83-01 | 20 μl | (500 nM) | TOCRIS(cat: 2939) |
| FGF2 | 20 | nG/mL | Peprotech(cat: 100-18B) |
| PGE2 | 10 | nM | TOCRIS (cat: 2296) |
| Y27632 | 10 | uM | Sigma (129830-38-2) |
| Total | 20 | mL | |

C) Growth Media for Growing Mini-Cancers:

| | | | Cat |
|---|---|---|---|
| Basic Media | 7 | mL | As above |
| HGF | 10 uL | (F Conc: 25 ng/uL) | Peprotech (cat: 100-39H) |
| FSK | 10 uL | (10 uM) | TOCRIS(cat: 1099) |
| FGF10 | 10 uL | (100 ng/mL) | Peprotech(cat: 100-26) |
| Gastrin I | 2 uL | (10 nM) | Sigma (10047-33-3) |
| NAC | 40 uL | (1.25 mM) | Sigma (A7250) |
| B27 supplement | 400 | uL | Gibco Ref: 12587-010 (50X) |
| N2 | 800 | uL | Gibco Ref: 17502-048 (100X) |
| Nicotinamide | 400 uL | (10 mM) | Sigma (N3376) |
| Wnt3A-conditioned medium | 6 mL | (30%) | Made in the lab - 293T |
| | | | cells (purchased from |
| | | | ATCC) |
| R-spondin1-conditioned | 4 mL | (20%) | Made in the lab - 293T |
| medium | | | cells (purchased from |
| | | | Trevigen) - HA-R- |
| | | | Spondin1-Fc293T) |
| EGF | 2 uL | (50 ng ml⁻¹) | Sigma(62253-63-8) |
| Noggin conditioned medium | 100 ng/mL | (2 uL) | Peprotech |
| A83-01 | 20 μl | (500 nM) | TOCRIS(cat: 2939) |
| FGF2 | 20 | nG/mL | Peprotech(cat: 100-18B) |
| PGE2 | 10 | nM | TOCRIS (cat: 2296) |
| Total | 20 | mL | |

### Example 2

### Steps to Plating Mini-Cancers for High Throughput Drug Testing

Take out Matrigel from −20 C and keep at 4 C overnight. Also keep pipette tips at 4 C overnight. Count cells. Mix cells with Matrigel so that you have 200 cells per uL of Matrigel. Take a Falcon—Ref: 35321996 96-well plate and place 2.5 uL of Matrigel+cells per well. Plate 2 rows, then stop. Wait till the Matrigel is solid. Then add 100 uL of growth passage media. Then continue till all plate is filled up with cells. Place plate into incubator. Take picture with white light. After 4 days replace the media with media containing drugs (90 uL media+10 uL drug work solution). Use a multi-pipette.

All references, including publications, patent applications, and patents, cited herein are hereby incorporated by reference to the same extent as if each reference were individually and specifically indicated to be incorporated by reference and were set forth in its entirety herein.

### Human Tissue

Fresh human CCA and matched histologically normal liver tissue were obtained from the Johns Hopkins Hospital (JHH), under an Institutional Review Board (IRB) approved informed consent. A diagnosis of CCA was performed by JHH pathologists, in accord with current standards in the field.

### Cell Lines

L Wnt-3A cells (Cat: CRL-2647™, ATCC, Manassas, Va.), as well as HA-R-Spondin1-Fc293T (Trevigen, Gaithersburg, Md.) cells were grown in DMEM medium supplemented with 10% fetal bovine serum (FBS) and antibiotics, and maintained in 37° C. humidified incubators, supplied with 5% CO2. Mycoplasma tests were performed weekly and only mycoplasma-free cells were utilized for experiments.

### Conditioned Medium

Conditioned media from L Wnt-3A and HA-R-Spondin1-Fc293T was obtained as previously described.

### PDO Medium

Media reported previously for normal liver organoids was adapted for use in the current study.

Establishment of PDOs from Human CCA and Matched Histologically Normal Liver

Resection liver tissue (CCA, HCC or normal liver) obtained from patients was minced in small pieces measuring approximately 9 mm³. Tissue was then rinsed 3 times with DMEM supplemented with 1% FBS at 4° C. in 50 mL Falcon tubes. The tissue was then dissociated with collagenase (2 mG/mL, Sigma) supplemented with DNAse 1 (0.1 mG/mL in DMEM, Sigma) at 37° C. Digestion was stopped by adding cold DMEM supplemented with 1% FBS. Cells were filtered, washed by centrifugation and then counted. After mixing with growth factor reduced Matrigel (Corning, Cat#: 356231, Tewksbury, Mass.), cells were seeded into 24-well plates. After the matrigel solidified within 15 min, warm organoid culture medium was added.

### PDOs Xenograft Establishment and Character

Animal experiments were performed under Johns Hopkins IACUC approved protocol, 500,000 PDO cells were mixed at 1:1 with Matrige and were subcutaneous injected into the flank of NSG mouse.

### Immunohistochemistry and Immunofluorescence

Fresh tissues as well as PDOs in culture were embedded into optimal cutting temperature compound (OCT) at −80° C. Tissue and PDO sections (8 uM) were prepared and stained. We utilized the following primary antibodies: LGRS (stem cell marker Cat#: AP2745f, Abgent, San Diego, Calif.), SOX9 (stem cell marker—Cat#: 82630, Cell signaling, Danvers, Mass.), CK7 (biliary epithelium marker—Cat#: ab9021, Abcam, Cambridge, Mass.) and CK19 (biliary epithelium markers—Cat#: MAB3238 Millipore, Danvers, Mass.), EPCAM (epithelial marker—Cat#: 2929, Cell Signaling, Cell signaling, Danvers, Mass.), Ki67 (cell proliferation—Cat#: 550609, BD San Jose, Calif.)). Alexa Fluor dye-conjugated secondary antibodies (Life Technologies, Carlsbad, Calif.). Microphotographs were obtained with a Zeiss laser scanning microscope (LSM 510). Hematoxylin and Eosin (H&E) staining (Sigma Aldrich, St. Louis, Mo.), mucicarmine staining for mucin (Cat#: ab150667, Abcam, Cambridge, Mass.) were performed following standard protocols. Pictures were obtained with a Zeiss Axio Observer Inverted Microscope.

### Whole-Exome Sequencing (WES) and Analysis

Total DNA was extracted from PDOs with Qiagen DNA kits. We used 1.5 uG DNA for WES with Agilent V5 capture probe set and sequenced by Illumina Hiseq2000 platform (BGI Hong Kong, China). The sequences of each sample were generated as 90/100 bp paired-end reads, and or each samples, around 10 Gb of unique sequences was generated. Standard quality control procedures were applied. Tumor-normal pair read alignment and variant identification, using SAMtools and GATK, were performed using standard parameters. A somatic variant was considered “present” in a tissue sample if it was present in at least 10% of reads. Using somatic coding (missense, nonsense, and canonical splice site) variations only, we performed phylogenetic tree estimation, using the maximum parsimony method with default settings in MEGA7.

### High-Throughput RNA Sequencing (RNA-Seq) and Analysis

Total RNA was isolated from PDOs with passage 4 in culture using Trizol following the manufacturer's instructions. 100 nG of total RNA were utilized for RNA-Seq using HIseq4000 SE50 (BGI Hong Kong, China) with 50 or 75 bp paired-end reads. An average of 43 million reads were generated for each sample. The gene expression level was quantified using the RSEM software package by BGI (Hong Kong, China).

### High-Throughput Drug Screening on CCA PDOs

A panel of 129 anti-cancer compounds (the NCI Approved Oncology Drugs Set VII) was utilized. PDOs were plated in 96-well or 384-well plates and cultured for 3 days. Next, media was changed with drug-containing media. The initial screen was performed at a concentration of 10 uM. After 4 days, viability was determined in each well with Cell TiterGlo (Cat#: G7572, Promega, Wis.) following the manufacturer's instruction. Fluorescent intensity was measured with a plate reader (Perkin-Elmer EnVision Plate Reader). Drugs that induced less than 50% viability were chosen for future studies. Inhibitory concentration 50 (IC50) experiments were performed at the following concentrations: 10 uM,1 uM, 100 nM, 10 nM and 1 nM.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for identifying a chemotherapy treatment for a subject comprising:
obtaining a biopsy from a subject;
establishing a cancer culture from the biopsy;
mixing the cancer culture with a matrix forming mini cancers;
growing the mini cancers;
dividing the mini cancers into one or more samples;
adding one or more agents separately or combined to the one or more samples;
analyzing the phenotypes of the mini cancers wherein the phenotypes are selected from the group of cell growth, invasion, or a combination thereof; and
identifying a chemotherapy treatment for the subject by choosing the one or more agents determined to stop growth of the mini cancers, inhibit growth of the mini cancers, and/or inhibit invasion of the mini cancers when compared to a reference mini cancer without the addition of one or more agent.

2. The method of claim 1 wherein the biopsy is of liver cancer and the cancer culture is created by mixing the biopsy with an establishing growth media comprising: HGF, FSK, FSK-10, Gastrin 1, NAC B27, N2, Nicotinamide, Wnt3A, R-spondin1, EGF, Noggin, A83-01, FGF2, PGE2, and Y27632.

3. The method of claim 2 wherein the cancer culture is then grown on a second growth media comprising: HGF, FSK, FGF10, Gastrin I, NAC, B27 supplement, N2, Nicotinamide, Wnt3A-conditioned medium, R-spondin1, EGF, Noffin conditioned medium, A83-01, FGF2, and PGE2.

4. The method of claim 1, wherein the biopsy is of a desmoplastic cancer and cells selected from the group comprising fibroblasts, endothelial cells, immune cells, other tumor-specific cells, or combinations thereof, are added to the cancer culture.

5. The method of claim 1, wherein the matrix comprises a polymer selected from the group consisting of collagen, gelatin, chitosan, heparin, fibrinogen, hyaluronic acid, chondroitin sulfate, pullulan, xylan, dextran, polyethylene glycol, derivatives thereof, or a combination thereof.

6. The method of claim 1, wherein the biopsy is of liver cancer.

7. The method of claim 1, wherein the biopsy is of pancreatic cancer.

8. The method of claim 1, wherein the step of identifying a chemotherapy treatment is based on a percentage of mini cancers killed by the agent when compared to a reference mini cancer substantially free of the one or more agents.

9. The method of claim 1, wherein the step of identifying a chemotherapy treatment is based on a percentage of mini cancers having inhibited growth when compared to a reference mini cancer substantially free of the one or more agents.

10. The method of claim 1, wherein the step of identifying a chemotherapy treatment is based on a percentage of mini cancers that are inhibited from invasion when compared to a reference mini cancer substantially free of the one or more agents.

11. The method of claim 1, wherein the samples are in the range of 10 to 4,000 samples.

12. A cell culture comprising HGF, FSK, FSK-10, Gastrin 1, NAC B27, N2, Nicotinamide, Wnt3A, R-spondin1, EGF, Noggin, A83-01, FGF2, PGE2, and Y27632.

13. A cell culture comprising: HGF, FSK, FGF10, Gastrin I, NAC, B27 supplement, N2, Nicotinamide, Wnt3A-conditioned medium, R-spondin1, EGF, Noffin conditioned medium, A83-01, FGF2, and PGE2.

14. A method of treating a patient having cancer comprising:
obtaining a biopsy from a subject with cancer;
establishing a cancer culture from the biopsy;
mixing the cell culture with a matrix forming mini cancers;
growing the mini cancers;
dividing the mini cancers into one or more samples;
adding one or more agents separately or combined to the one or more samples;
analyzing the phenotypes of the mini cancers wherein the phenotypes are selected from the group of cell growth, invasion, or a combination thereof;
identifying an agent for the subject by choosing the one or more agents determined to stop growth of the mini cancers, inhibit growth of the mini cancers, or inhibit invasion of the mini cancers when compared to a reference mini cancer without the addition of one or more agents; and
administering the one or more agents to the subject to treat the cancer.

15. The method of claim 14 wherein the process is repeated should the subject have a cancer recurrence after treatment.

16. The method of claim 15 wherein the subject is treated with a second one or more agent identified during the repeated process.

17. The method of claim 14 wherein the biopsy is of liver cancer and the cancer culture is created by mixing the biopsy with an establishing growth media comprising: HGF, FSK, FSK-10, Gastrin 1, NAC B27, N2, Nicotinamide, Wnt3A, R-spondin1, EGF, Noggin, A83-01, FGF2, PGE2, and Y27632.

18. The method of claim 17 wherein the cancer culture is then grown on a second growth media comprising: HGF, FSK, FGF10, Gastrin I, NAC, B27 supplement, N2, Nicotinamide, Wnt3A-conditioned medium, R-spondin1, EGF, Noffin conditioned medium, A83-01, FGF2, and PGE2.

19. The method of claim 15, wherein the cancer is a desmoplastic cancer and cells selected from the group comprising fibroblasts, endothelial cells, immune cells, other tumor-specific cells, or combinations thereof are added to the cancer culture.

20. The method of claim 15, wherein the matrigel comprises a polymer selected from the group consisting of collagen, gelatin, chitosan, heparin, fibrinogen, hyaluronic acid, chondroitin sulfate, pullulan, xylan, dextran, and polyethylene glycol as well as their derivatives or a combination thereof.
